(19) United States
(12) Patent Application Publication
(10) Pub. No.: US 2005/0176827 A1
(43) Pub. Date: Aug. 11, 2005

(54) Compositions and methods for glycogen synthesis

(76) Inventors: Steve S. Lee, Sandy, UT (US); Richard B. Hynson, Missoula, MT (US); Brent C. Ruby, Missoula, MT (US); Steven E. Gaskill, Missoula, MT (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/926,849

(22) Filed: Aug. 26, 2004

Related U.S. Application Data
(63) Continuation-in-part of application No. 10/434,444, filed on May 7, 2003.
(60) Provisional application No. 60/379,839, filed on May 10, 2002.
(60) Provisional application No. 60/498,717, filed on Aug. 28, 2003.

(51) Int. Cl. A61K 31/198

(52) U.S. Cl. 514/561

(57) ABSTRACT

A composition of bio-active compounds and methods for facilitating and supporting the metabolism and transport of glucose and carbohydrates into muscle cells, promoting muscle function and growth, promoting glycogen synthesis, enhancing glucose disposal, stimulating pancreatic beta cells, promoting metabolic recovery, promoting muscle recovery, promoting lean body mass, and promoting fat burning. Preferably, the composition of bio-active compounds includes a combination of 4-hydroxyisoleucine with at least one amino acid selected from the group consisting of arginine, aspartate, threonine, serine, glutamate, proline, glycine, alanine, cysteine, valine, methionine, isoleucine, leucine, tryptophan, phenylalanine, ornithine, lysine, histidine, gamma-amino butyrate and tyrosine. In one presently preferred embodiment of the present invention, the combination is derived, isolated, and/or extracted from fenugreek seeds. Methods for using a novel composition of bio-active compounds from fenugreek seed for facilitating and supporting the metabolism and transport of glucose and carbohydrates into muscle cells, promoting muscle function and growth, promoting glycogen synthesis, enhancing glucose disposal, stimulating pancreatic beta cells, promoting metabolic recovery, promoting muscle recovery, promoting lean body mass, and promoting fat burning are also disclosed, wherein methods comprise the steps of: (1) providing an effective amount of a composition of bio-active compounds derived, isolated, and/or extracted from fenugreek seeds; and (2) administering the composition to a human or animal.

1. RELATED APPLICATIONS

This application claims the benefit of U.S. Provisional Patent Application Ser. No. 60/498,717, filed Aug. 28, 2003, and entitled “COMPOSITIONS AND METHODS FOR GLYCOGEN SYNTHESIS” and co-pending U.S. patent application Ser. No. 10/434,444, filed May 7, 2003, and entitled “FENUGREEK SEED BIO-ACTIVE COMPOSITIONS AND METHODS FOR EXTRACTING SAME,” which claims priority to U.S. Provisional Patent Application Ser. No. 60/379,839, filed May 10, 2002, and entitled “BIO-ACTIVE FENUGREEK SEED COMPONENT EXTRACTION METHOD,” all of which are incorporated herein by reference.

### BACKGROUND

2. Field of the Invention

This invention relates to methods and compositions affecting metabolism, and more particularly, to novel compositions of bio-active components derived, isolated, and/or extracted from fenugreek seeds that promote glycogen synthesis, muscle function, the enhancement of glucose disposal, the stimulation of pancreatic beta cells to produce insulin, the transportation of glucose and carbohydrates in animals and humans, metabolism support and/or metabolic recovery, together with methods for using the same.

3. The Background Art

Fenugreek is one of the oldest medicinal herbs and is native to southeastern Europe, northern Africa, and western Asia, but is widely cultivated in other parts of the world. Fenugreek is known technically as Trigonella foenum-graecum, a member of the family Fabaceae, and commonly referred to as Greek hay. As appreciated by those skilled in the art, fenugreek is a legume and typically grows between two (2) to three (3) feet tall with light green leaves and small white flowers. A fenugreek seed pod may contain between ten (10) to twenty (20) small, flat, yellow-brown seeds. Typically, a plant seed is formed having a thick or hard outer coat called a testa and often referred to as a seed coat. The inner portion of the seed coat contains a plant embryo and a nutritive tissue called endosperm, which surrounds the embryo. As the fenugreek seed embryo matures, it consumes endosperm. Fenugreek seeds often have a pungent aroma and may have a bitter taste, which is said to be similar to celery.

Fenugreek has long been used as a medicinal herb and culinary additive in both Asia and the Mediterranean. It is believed that the seed of the fenugreek plant contains many active compounds with pharmaceutical applications such as, for example, iron, vitamin A, vitamin B, vitamin C, phosphates, flavonoids, saponins, trigonelline and other alkaloids. Fenugreek has been taken as a stomach tonic and as a treatment for abdominal ailments. Western scientific research has provided insight into the chemical analysis of fenugreek seeds, together with the extraction of 4-hydroxyisoleucine from Fenugreek seeds, and has suggested some clinical utilities of fenugreek.

Sir L. Fowden conducted research into the analysis of fenugreek. He taught the isolation and purification of 4-hydroxyisoleucine from fenugreek and claimed that it is the principal unbound amino acid contained in the fenugreek seed. (See, Fowden et al, Phytochemistry, 12:1707, (1973).) Further investigation of the prior art suggests that the amino acids of fenugreek seeds may have nutritional value. (See, Sauvaire et al, Nutr Rep Int, 14:527 (1976).)

Other compounds have also been isolated from fenugreek seeds. In addition to the major isomers (2S, 3R, 4S)-4-hydroxyisoleucine, minor isomers 4-hydroxyisoleucine, and amino acids (lysine, histidine, and arginine) have been isolated.

As appreciated by those skilled in the art, the major isomer (2S, 3R, 4S) is presently interesting with respect to experimental evidence indicating its ability to stimulate glucose-induced insulin secretion in micromolar concentrations through a direct effect on pancreatic beta cell stimulation in a glucose dependent manner. Moreover, 4-hydroxyisoleucine has been shown to interact and induce additive insulinotropic effects (i.e., stimulating or affecting the production and activity of insulin, only in the presence of supranormal glucose concentrations). (See, Sauvaire et al, Diabetes, 47:206 (1998).)

Investigation of the prior art also discloses clinical studies to investigate the use of subfractions of Fenugreek in conditions of hyperglycemia, glucosuria and hyperlipidemia which have been performed on rats, dogs and human pancreatic tissue. (See, Shani et al, Arch Intern Pharmacodyn Ther, 210:27 (1974); Ribes et al, Ann Nutr Metab, 28: 37 (1984); Valette et al, Athersclerosis, 50:105 (1984); Madar, Nutr Rep Int, 29:1267 (1984).)

As appreciated by those skilled in the art, clinical studies directed to conditions of hyperglycemia, as well as other conditions involving the metabolism of carbohydrates, have only investigated 4-hydroxyisoleucine as an effector of insulin-mediated or insulin-dependent pathways. The available prior art do not teach or suggest Fenugreek and/or 4-hydroxyisoleucine compositions which may work synergistically or independently from insulin or insulin-mediated pathways. More particularly, there are no prior art teachings or suggestions that 4-hydroxyisoleucine may affect the body by an insulin-independent mechanism. Stimulation of non-insulin mediated pathways may be desirous for targeting the utilization of carbohydrates in certain organ systems, (e.g., muscles, liver, etc.). Likewise, it may be desirous to avoid the general and/or systemic effects that may occur by stimulating the pancreas to produce and secrete insulin.

Studies have also shown that the natural analogue of 4-hydroxyisoleucine is more effective as an antidiabetic agent than a synthetic version. There is, therefore, a suggestion that the therapeutic effects of 4-hydroxyisoleucine are best obtained from extracts of the fenugreek seed.

During the aforementioned research and clinical investigations, those skilled in the art developed crude methods for extracting 4-hydroxyisoleucine from fenugreek seeds and examined the use of 4-hydroxyisoleucine extracts in confectionary applications and in the treatment of diabetes and cholesterol in mammals. However, these previous clinical investigations have not investigated the use of fenugreek seed extracts for the use of glycogen synthesis in the muscle, liver, and other tissues in mammals.

While past research by those skilled in the art has attempted to maximize the insulinemic (i.e., increased concentration of insulin in blood) effect of dietary carbohydrate by including supplemental protein and/or essential amino acids, it is unclear whether amplifying the circulating insulin concentration will have an additive effect on muscle glucose uptake and the overall rate of muscle glycogen re-synthesis. Moreover, in conditions requiring significant or prolonged muscle activity resulting in the depletion of muscle glycogen stores, the prior art has not heretofore contemplated the exogenous administration of specific fenugreek compositions and methods for stimulating glycogen synthesis. Therefore, as readily appreciated by those skilled in the art, novel compositions of bio-active compounds derived, isolated, and/or extracted from fenugreek seed and methods for using the same to stimulate glycogen synthesis in mammals would be a significant advancement in the art. Such novel compositions and methods are disclosed and taught herein.

### BRIEF SUMMARY AND OBJECTS OF THE INVENTION

A primary object of the present invention is to provide novel compositions and methods of bio-active compounds derived, isolated, and/or extracted from fenugreek seeds for promoting glycogen synthesis and/or re-synthesis in mammals.

It is another object of the present invention to provide novel compositions and methods of bio-active compounds derived, isolated, and/or extracted from fenugreek seeds which are capable of enhancing glycogen synthesis in muscles.

It is also an object of the present invention to provide novel compositions and methods of bio-active compounds derived, isolated, and/or extracted from fenugreek seeds which are capable of enhancing glycogen synthesis in the hepatic system, gastrointestinal system, renal system, nervous system, cardiopulmonary system, and other areas of the body.

It is a further object of the present invention to provide novel compositions and methods of bio-active compounds derived, isolated, and/or extracted from fenugreek seeds which are capable of stimulating the function of glucose transport factor 4.

In addition, it is an object of the invention to provide novel compositions and methods of bio-active compounds derived, isolated, and/or extracted from fenugreek seeds which may be administered through the oral, parenteral, sublingual, topical, transdermal, intramuscular, intranasal, and inhalation routes.

It is a further object of the present invention to provide novel compositions and methods of bio-active compounds derived, isolated, and/or extracted from fenugreek seeds which may be provided in a dietary food or nutritional supplement form, such as a juice, liquid, tablet, capsule, granule, pellet, powder, lozenge, or bar.

It is a still further object of the present invention to provide novel compositions of bio-active compounds derived, isolated, and/or extracted from Fenugreek seeds which may be delivered and/or administered using any pharmaceutical delivery form, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, and the like.

It is another object of the present invention to provide novel compositions of bio-active compounds derived, isolated, and/or extracted from Fenugreek seeds which may be delivered and/or administered using any nutraceutical delivery form, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, and the like.

In addition, it is an object of the present invention to provide novel compositions of bio-active compounds derived, isolated, and/or extracted from Fenugreek seeds which may be delivered and/or administered using any functional food delivery form, for example and not by way of limitation, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Also, it is an object of the present invention to provide novel compositions of bio-active compounds derived, isolated, and/or extracted from Fenugreek which may be used in combination with other amino acids, botanicals, herbals, nucleotides, nutraceuticals, nutrients, pharmaceuticals, proteins, vitamins, and the like.

It is also an object of the present invention to provide novel compositions and methods of bio-active compounds derived, isolated, and/or extracted from fenugreek seeds which are economical to produce.

Additionally, it is an object of the present invention to provide novel compositions and methods of bio-active compounds derived, isolated, and/or extracted from fenugreek seeds which are anti-hyperglycemic.

It is a further object of the present invention to provide novel compositions and methods of bio-active compounds derived, isolated, and/or extracted from fenugreek seeds which may be combined with carbohydrates, muscle growth supplements, and/or dietary supplements for the promotion of glycogen synthesis.

It is a still further object of the present invention to provide novel compositions and methods of bio-active compounds derived, isolated, and/or extracted from fenugreek seeds which are capable of independently stimulating glucose transport proteins and facilitating the transport of glucose into muscle cells.

It is also an object of the present invention to provide novel compositions and methods of bio-active compounds derived, isolated, and/or extracted from fenugreek seeds which work synergistically with insulin to stimulate glucose transport proteins and facilitate the transport of glucose into muscles.

Additionally, it is an object of the present invention to provide novel compositions and methods of bio-active compounds derived, isolated, and/or extracted from fenugreek seeds which facilitate glucose absorption from the gastrointestinal tract.

It is a further object of the present invention to provide novel compositions and methods of bio-active compounds derived, isolated, and/or extracted from fenugreek seeds which enhance protein synthesis in response to vigorous muscle work.

It is a still further object of the present invention to provide novel compositions and methods of bio-active compounds derived, isolated, and/or extracted from fenugreek seeds which are capable of enhancing glycogen synthesis and storage in impaired muscle and/or in animals with neuromuscular disorders.

It is also an object of the present invention to provide novel compositions and methods of bio-active compounds derived, isolated, and/or extracted from fenugreek seeds which enhance glucose disposal from the blood.

Additionally, it is also an object of the present invention to provide novel compositions and methods of bio-active compounds derived, isolated, and/or extracted from fenugreek seeds which stimulate pancreatic beta cells to produce insulin.

It is a further object of the present invention to provide novel compositions and methods of bio-active compounds derived, isolated, and/or extracted from fenugreek seeds which may be combined with carbohydrates, insulin, and/or insulin response modifiers which stimulate pancreatic beta cells to produce insulin or enhance insulin sensitivity and function.

It is a still further object of the present invention to provide novel compositions and methods of bio-active compounds derived, isolated, and/or extracted from fenugreek seeds which facilitate metabolic recovery in a mammal following exercise training, conditioning, and/or enhancement of athletic performance.

It is also an object of the present invention to provide novel compositions and methods of bio-active compounds derived, isolated, and/or extracted from fenugreek seeds which may be combined with carbohydrates and/or protein and administered to a mammal immediately following (e.g., within 30 minutes) muscular activity to enhance glycogen synthesis and promote recovery.

Consistent with the foregoing objects, and in accordance with the invention as embodied and broadly described herein, one presently preferred embodiment of the present invention comprises novel compositions and methods of bio-active compounds derived, isolated, and/or extracted from fenugreek seeds which may be used to facilitate and support metabolism and transportation of glucose and other carbohydrates and to promote glycogen synthesis. Specifically, one presently preferred embodiment of a composition of bio-active compounds derived, isolated, and/or extracted from fenugreek seeds may work either synergistically with insulin, or independent, to stimulate GT-4 receptors located on muscle cells. The composition, so isolated, is generally comprised of amino acids and proteins.

In one presently preferred embodiment of the present invention, particular emphasis is placed on a composition of bio-active compounds derived, isolated, and/or extracted from fenugreek seeds which includes, for example, 4-hydroxyisoleucine and one or more of the following compounds: arginine, aspartate, threonine, serine, glutamate, proline, glycine, alanine, cysteine, valine, methionine, isoleucine, leucine, tryptophan, phenylalanine, ornithine, lysine, histidine, and gamma-aminobutyrate. In yet another presently preferred embodiment, a composition of bio-active compounds derived, isolated, and/or extracted from fenugreek seeds, together with other components, may be combined with glucose, another carbohydrate, muscle growth supplements, insulin, and/or insulin response modifiers for the promotion of glycogen synthesis.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a graph illustrating the changes in insulin and glucose concentrations in response to the administration of oral glucose and an experimental feeding containing either placebo or a novel composition of bio-active compounds derived, isolated, and/or extracted from fenugreek seeds;

FIG. 2 is a graph illustrating the changes in serum glucose concentrations in response to the administration of oral glucose and experimental feedings containing either placebo or a novel composition of bio-active compounds derived, isolated, and/or extracted from fenugreek seeds;

FIG. 3 is a graph illustrating the changes in insulin concentration in response to the administration of oral glucose and experimental feedings containing either placebo or a novel composition of bio-active compounds derived, isolated, and/or extracted from fenugreek seeds;

FIG. 4 is a bar graph illustrating the total insulin area under the curve for 0-45 minutes in response to the administration of oral glucose and experimental feedings containing either placebo or a novel composition of bio-active compounds derived, isolated, and/or extracted from fenugreek seeds;

FIG. 5 is a bar graph illustrating the percentage (%) insulin increase by time period in response to the administration of oral glucose and experimental feedings containing either placebo or a novel composition of bio-active compounds derived, isolated, and/or extracted from fenugreek seeds;

FIG. 6 represents a graph showing the changes in blood glucose in trained male cyclists in response to the administration of oral glucose and experimental feedings containing either placebo or a novel composition of bio-active compounds derived, isolated, and/or extracted from fenugreek seeds;

FIG. 7 represents a graph showing the changes in serum insulin in the group of trained male cyclists, as referenced in FIG. 6, in response to the administration of oral glucose and experimental feedings containing either placebo or a novel composition of bio-active compounds derived, isolated, and/or extracted from fenugreek seeds; and

FIG. 8 represents a graph showing the changes in muscle glycogen in the group of trained male cyclists, as referenced in FIGS. 6 and 7, in response to the administration of oral glucose and experimental feedings containing either placebo or a novel composition of bio-active compounds derived, isolated, and/or extracted from fenugreek seeds.

### DESCRIPTIONS OF THE PREFERRED EMBODIMENTS

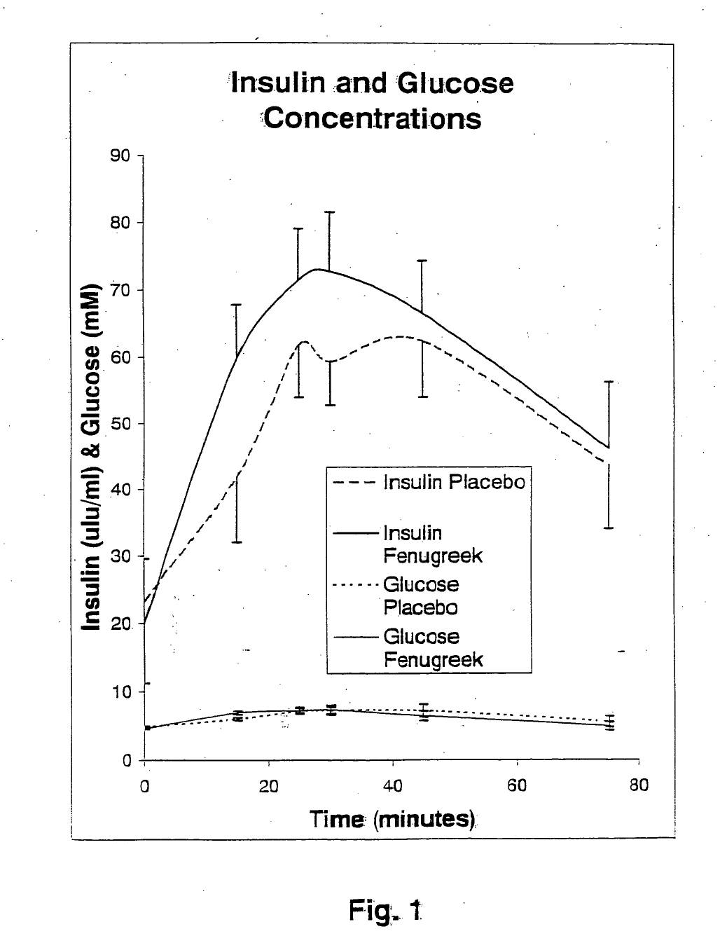

It will be readily understood that the components of the present invention, as generally described and illustrated in the Figures herein, could be arranged and designed in a wide variety of different configurations. Those of ordinary skill in the art will, of course, appreciate that various modifications to the details herein may be made without departing from the essential characteristics of the invention, as described. Thus, the following more detailed description of the embodiments of the compositions and methods of the present invention, as represented in FIGS. 1 through 8, is not intended to limit the scope of the invention, as claimed, but it is merely representative of the presently preferred embodiments of the invention.

In an effort to establish a background relative to the physiology and biochemical processes concerning the present innovative technology and to provide definitional support for various terms that may be used in the present application, the following technical comments and review are provided as a basis for understanding the implications of the present invention.

Carbohydrates serve an important role in the storage of energy in mammals. Carbohydrates are generally defined as any of a group of organic compounds that includes sugars, starches, celluloses, and gums. Carbohydrates are often found in the dietary intake of animals. They may be produced by photosynthetic plants and may contain only carbon (C), hydrogen (H), and oxygen (O), usually in the ratio 1:2:1, respectively.

The term saccharide is sometimes used to describe a sugar. A saccharide may include any of a series of compounds consisting of carbon, hydrogen, and oxygen in which the atoms of the latter two elements, H and O, are in the ratio of 2:1, respectively, for example, C₆H₁₀O₅ and C₅H₁₀O₅ Saccharides may also be classified according to how many units or components they contain. A monosaccharide is the simplest form of saccharide and includes those carbohydrates which cannot be hydrolyzed into a simpler form. Monosaccharides may include organic compounds with between three (3) and nine (9) carbon atoms. Disaccharides may be defined as compounds which, upon hydrolysis, yield two (2) monosaccharides that may be the same or different. Oligosaccharides may be defined as compounds which, upon hydrolysis, yield between three (3) and six (6) monosaccharide units that may be the same or different. A polysaccharide may be defined as compounds which, upon hydrolysis, yield more than six (6) monosaccharide units that may be the same or different.

Several saccharides have important physiological roles in the body. Many monosaccharide sugars function as the basic building blocks for more complex biomolecules such as proteins and nucleic acids. Five carbon and six carbon sugars (known as pentoses and hexoses, respectively) serve many of these important physiological roles. Pentoses may include, ribose, ribulose, arabinose, xylose, lyxose, and xylulose. Hexoses may include, glucose, fructose, galactose and mannose.

Glycogen is an important carbohydrate occurring in mammals. As appreciated, glycogen is a polysaccharide containing a plurality of glucose residues connected though bonds. Glycogen is commonly stored by the body in the hepatic system (i.e., liver) and in muscle tissue, but may be found in other areas of the body including the gastrointestinal system (e.g., intestines), renal system (i.e., kidney), nervous system (e.g., brain, nerves), and cardiopulmonary system (i.e., heart, lungs). When the body requires energy, glycogen may be broken into smaller units and made available for various biochemical and homeostatic processes.

As appreciated by those skilled in the art, muscle function (i.e., contraction, relaxation, growth) is a complex physiological mechanism. Muscle contraction may be defined as the shortening and thickening of a muscle or muscle fiber in response to a voluntary or involuntary stimulus. Muscle relaxation may be defined as the lengthening of muscles or muscle fiber that occurs upon muscle deactivation. Muscle growth may be defined as an increase in the size and/or dimension of muscle or muscle fiber.

Mammals may have three main types of muscle: skeletal muscle, cardiac muscle, and smooth muscle. Skeletal muscle attaches to bones and supports the skeleton. Cardiac muscle is found in the heart and is responsible for pumping blood. Smooth muscles may line hollow cavities, visceral organs, and tubes (e.g., circulatory vessels) within mammals. Skeletal and cardiac muscle may have a characteristic banding appearance and may sometimes be referred to as striated muscle. Striated muscle is generally composed of several muscle fibers, which in turn are composed of several myofibrils. Myofibrils utilize an overlapping pattern of thick and thin filaments arranged into units known as sarcomeres. Thick filaments may be almost entirely composed of myosin protein. Thin filaments may be almost entirely composed of actin protein. Portions of myosin protein may extend from the thick filaments toward thin filaments and may form cross-bridges. Smooth muscle may also contain thick and thin filaments.

Muscle contraction may follow a sliding filament mechanism. In this mechanism, a cross-bridge cycle may comprise four (4) steps. First, a cross bridge may attach to an actin protein on a thin filament. Second, a cross-bridge may move, causing the thin filament to move. Third, a cross-bridge may detach from actin protein. Finally, a cross-bridge may move to a new position. Tropomyosin protein is located on actin near the binding site for myosin cross-bridges. In order for binding to occur, tropomyosin may be moved out of the binding-site by troponin, whereas troponin is activated by intracellular calcium to move tropomyosin.

The energy molecule, adenosine 5′-triphosphate (ATP), may be required for muscle contraction. An ATP molecule may be hydrolyzed to provide the energy for cross-bridge movement (i. e., second step) and may be hydrolyzed to allow detachment of the cross-bridge from actin (i.e., third step). ATP may also be required to sequester calcium ions into the sarcoplasmic reticulum in order to promote muscle relaxation. As appreciated, muscle function may require significant energy requirements (e.g., in the form of ATP).

ATP is an important form of energy for the body and many biochemical pathways utilize ATP. ATP may be generated in vivo though the glycolysis, citric acid (i.e., krebs cycle), and oxidative phosphorylation (i.e., respiratory chain oxidation-reduction reactions) cycles. As appreciated, these cycles may all be linked to the conversion of glucose-6-phosphate from a glucose-1-phosphate molecule.

Muscle tissues undergoing contraction and relaxation often may require rapid access to carbohydrates, more specifically, glucose-1-phosphate molecules, for use as a source of energy and muscles are known to contain glycogen for this purpose. The mass and volume of muscle tissue may sometimes limit the amount of glycogen that may be stored in muscle. In conditions of muscle performance, such as athletic performance or other metabolic conditions requiring muscle activity, glycogen stores may be rapidly depleted. Mammals have developed biochemical mechanisms for rebuilding or re-synthesizing glycogen stores in muscle and other areas of the body. However, many of these mechanisms are not completely understood.

It has been established that regular carbohydrate feedings are required soon after glycogen depleting muscle work in order to maximize the rates of muscle glycogen resynthesis. It is also clear that glucose uptake by skeletal muscle is most apparent in response to the translocation (i.e., removal of something from one place to another) of the glucose transporter type 4 (GT-4 or Glut-4) via contractile activity and to some degree, dietary nutrient stimulated insulin release. Stated another way, it appears that the Glut-4 protein may be responsible for transporting glucose molecules from the blood across the cell membrane and into the cytosol of skeletal muscle cells. Moreover, this transport process may be stimulated by any number of factors including, but not limited to, elevated blood glucose levels, insulin, and contractile activity in muscles. However, previous research has not established a clear link between elevated post-exercise insulin release and enhanced muscle glucose uptake.

The disposition of dietary nutrients in mammals following digestion and absorption is commonly referred to as intermediary metabolism and sometimes shortened to metabolism. Intermediary metabolism is a collection of numerous biochemical pathways and processes that impact every cell and organ in the body. Metabolic pathways may be defined as being anabolic (i.e., involved in the synthesis of compounds constituting body structure; “building processes”), catabolic (i.e., involved in oxidative processes which release free-energy for use in other reactions and processes; “break-down processes”), and amphibiotic (i.e., involved in multiple functions and multiple processes).

The dietary intake and energetic needs of the body are largely responsible for determining the type of metabolic pathways that are active and dormant at a given time. The dietary intake of mammals may be processed through the small intestine and may be roughly divided by the liver and other areas of the body into carbohydrate, lipid, and protein components. A major component of each of these categories are glucose, triacylglycerol, and amino acids, respectively. Carbohydrate metabolism is primarily channeled through the use of glucose molecules.

A glucose molecule derived from the diet may be converted to glucose phosphate and shunted into several different biochemical pathways, depending on the needs, requirements, or conditions of the body. For example, glucose phosphate may be directed into the pentose phosphate pathway or may be broken down into pyruvate and other compounds through a process known as glycolysis. Glycolysis may be accomplished in anaerobic or aerobic conditions. In aerobic conditions, glucose molecules may enter the citric acid cycle to generate free-energy for use in the body. In this catabolic process, glucose serves as a major fuel source for the body.

In conditions where the body may have sufficient dietary intake and basal energy (i.e., something essential for maintaining the fundamental vital activities of an organism) needs may be minimal, anabolic metabolism may occur. Under these conditions, glycogenesis (an anabolic process) may be initiated in the cells of the liver, muscle, intestine, kidney, and other organs. Glycogenesis is the conversion of excess glucose into glycogen. As readily known in the art, glycogen is a polysaccharide containing multiple glucose molecules or residues which are connected by glucosidic links or bonds.

Synthesis of a glycogen chain (i.e., glycogenesis) is initiated by the phosphorylation (i.e., addition of a phosphate group to an organic compound) of a glucose molecule. This reaction may be catalyzed by the hexokinase enzyme in the muscle and the glucokinase enzyme in the liver. The resulting compound is called glucose-6-phosphate. The enzyme phosphoglucomutase may then move the phosphate from the 6 position to the 1 position on the glucose molecule. The compound, glucose-1-phosphate, may then be combined with uridine triphosphate to form uridine diphosphate glucose (UDPGlc).

UDPGlc generally serves as a primer for the propagation of the glycogen chain. In the presence of the enzyme, glycogen synthase, UDPGlc may react with the terminal residue of another glycogen chain to form a glycosidic bond. In this process, uridine diphosphate may be generated. Other enzymes may also be involved in branching the glycogen chain at specific intervals.

Glycogen catabolism may be commonly referred to as glycogenolysis and a key enzyme in this process is phosphorylase. Phosphorylase may insert a phosphate molecule at the glycosidic linkage thus creating a glucose-1-phosphate molecule. This molecule may then be utilized by many biochemical processes requiring the generation of free-energy.

The control of glycogen metabolism may primarily be accomplished through regulation of the principal enzymes, glycogen synthase, and phosphorylase. The regulation process is known to be very complex and may involve several allosteric mechanisms (i.e., feedback mechanisms). These feedback mechanisms are often controlled by hormones, neurotransmitters, and other chemical signals. Feedback mechanisms controlling glycogen metabolism are important to the maintenance of homeostasis (i.e., activity of an organism or cell to maintain internal equilibrium by adjusting its physiological processes) and prevention of damage to cells, tissues, and organs.

As described hereinabove, athletic performance or other conditions requiring significant muscle activity (i.e., muscle contraction, relaxation, growth), may generate body energy requirements that exceed the amount of glucose available from normal dietary intake. The body, especially the skeletal muscles, may signal for or stimulate the activity of phosphorylase to liberate a plurality of glucose-1-phosphate molecules for instant energy needs. Likewise, in conditions where energy needs are minimal, the body may signal or stimulate the activity of glycogen synthase to produce or replenish glycogen stores from available dietary sugars.

Many disorders of carbohydrate metabolism are well-known in the art. Some of these conditions, as discussed below and others, may have significant impact on the ability to store or utilize glycogen.

The term “diabetes,” when used alone, most commonly refers to diabetes mellitus. Diabetes mellitus generally refers to as a disorder of carbohydrate metabolism wherein sugars cannot be properly broken down and utilized by the body. The dysfunction in carbohydrate metabolism may generally be associated with a lack of adequate production of insulin. As appreciated by those skilled in the art, insulin is produced by specialized pancreatic cells known as beta cells of islets of Langerhans. Insulin is secreted by the pancreas and moves throughout the body to regulate sugar metabolism and, more particularly, to regulate glucose levels in the bloodstream. Specifically, insulin interacts with several types of cell surface receptors. One such family of receptors is called glucose transport (GT) receptors. The term receptor is often used interchangeably the term protein. At least seven (7) subtypes of the GT receptor family, designated as 1-7 (GT-1-7), have been identified by those skilled in the art.

Many non-diabetic individuals may experience nutritional and metabolic deficiencies during their lifetime which may significantly impact the proper metabolism of glucose and carbohydrates. For example, some individuals may develop a resistance to insulin. These individuals generally have difficulty transporting and metabolizing sugars, but not to the level of an individual with diabetes mellitus. One such condition, generally known as “Syndrome X,” may be characterized by elevated blood glucose levels and fat deposition, especially in the abdominal area. It is contemplated that “Syndrome X” may include any number of collected conditions which may be impacted by impairments in the transport and metabolism of sugars, especially glucose, and carbohydrates. For example, and not by way of limitation, “Syndrome X” may include obesity, hyperinsulinemia (i.e., high blood insulin levels), hypertension (i.e., high blood pressure), dyslipidemia (i.e., abnormal levels of fats and lipids in blood), insulin resistance, and/or impaired glucose tolerance.

A condition of elevated blood glucose level is sometimes referred to as hyperglycemic or hyperglycemia. In many non-diabetic individuals experiencing nutritional and metabolic deficiencies, it may be advantageous to facilitate and support the proper transport and metabolism of glucose and other carbohydrates in the body. The facilitation and support of glucose and carbohydrate metabolism may be accomplished with the administration of compositions that work synergistically with existing insulin in the regulation of glucose and other carbohydrate metabolism or, in the alternative, through the administration of compositions that have an effect independent of insulin in the regulation of glucose and other carbohydrate metabolism.

Therapies for diabetes, Syndrome X and other nutritional and metabolic dysfunction may involve the administration of exogenous insulin and/or one or more insulin response modifiers. Insulin response modifiers may work to enhance insulin sensitivity or may enhance insulin function in clearing carbohydrates from blood. Insulin response modifiers may include insulin secretagogues (i.e., a substance that stimulates secretion), biguanides, alpha-glucosidase inhibitors, and thiazolidinediones. Insulin secretagogues stimulate the pancreas, and more specifically, beta cells of islets of Langerhans, to secrete insulin. An important group of insulin secretagogues includes sulfonylurea compounds. Sulfonylurea compounds may comprise tolbutamide, acetohexamide, tolazamide, chlorpropamide, glyburide, glipizide, and glimepiride.

As readily known by those skilled in the art, biguanides enhance glucose uptake by peripheral muscle and inhibit glucose release by the liver. Biguanides may include metformin. Alpha-glucosidase inhibitors may block the action of alpha-glucosidase enzymes which hydrolyze complex starches into oligo- and monosaccharides and glucose. Blocking these enzymes may reduce post-prandial (i.e., period after eating) plasma glucose levels. Alpha-glucosidase inhibitors may include acarbose and miglitol. Thiazolidinediones typically decrease insulin resistance modifying enzyme systems, including the P-Par gamma system.

In those mammals who may be suffering from an acute and/or critical illness involving one or more organ systems, there may be significant difficulty in generating and/or maintaining glycogen stores in muscles and in the liver. Glycogen storage may also be affected in those mammals suffering from trauma or surgical procedures. In addition, glycogen storage may be affected in those mammals suffering from neuromuscular disorders (e.g., myopathies, neuropathies, motor neuron disease, myasthenia gravis) and inherited disorders (e.g., glycogen storage disease).

Fenugreek is recognized and has been known to have effects on lowering blood sugar and blood lipid levels. However, it was not until about thirty (30) years ago that systematic scientific investigations of fenugreek were initiated and subsequently, 4-hydroxyisoleucine was identified as a component of fenugreek. 4-Hydroxyisoleucine is usually classified as an amino acid compound and has the following general formula:

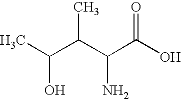

As known in the art, amino acids may be defined as organic acids containing both an amino and carboxylic acid functional group, and which a portion of the nonacid hydrogen has been replaced by one or more amino groups. An amino acid may therefore have both basic and acidic properties. More than three hundred (300) amino acids are known to occur in nature, however, only twenty (20) amino acids are presently used in the in vivo synthesis of protein chains.

These twenty (20) amino acids have the optical absolute configuration of L-glyceraldehyde and are therefore labeled as L-α amino acids. The L-α amino acids include alanine, arginine, asparagine, aspartic acid (also referred to as aspartate), cysteine, glutamic acid (also referred to as glutamate), glutamine, glycine, histidine, isoleucine, leucine, lysine, methionine, phenylalanine, proline, serine, threonine, tryptophan, tyrosine, and valine.

Moreover, nine (9) of the twenty (20) amino acids cannot be manufactured in vivo by animals and must be supplied through the hydrolysis of dietary protein. These nine (9) amino acids may be defined as essential amino acids and include, arginine, isoleucine, leucine, lysine, methionine, phenylalanine, threonine, tryptophan, and valine.

Certain L-α amino acids have chemical and physical properties based on their respective side chains. For example, the side chains of isoleucine, leucine, and valine all contain branched-chain aliphatic groups. These three (3) amino acids are therefore commonly referred to as branched-chain amino acids (BCAA's). Other amino acids contain unique functional groups including: hyrdoxylic groups (e.g., serine, threonine, tyrosine); sulfur atoms (e.g., cysteine, methionine); acid groups or their amides (e.g., aspartic acid, asparagine, glutamic acid, glutamine); basic groups (e.g., arginine, lysine, histidine); aliphatic groups (e.g., alanine, glycine); and aromatic rings (histidine, phenylalanine, tyrosine, tryptophan).

Amino acids may serve many important roles in the homeostasis and physiological functions in animals. BCAA's are important to muscle growth and may account for the most common amino acids in muscle tissue. They are also important to the synthesis of neurotransmitters (e.g., tyrosine, tryptophan) for the nervous system, and in the case of some amino acids, actually function as neurotransmitters (e.g., gamma-aminobutyrate, glutamate, glycine). Amino acids containing basic groups (i.e., arginine, lysine, histidine) are also important to muscle growth. These amino acids may serve as a precursor to growth hormone and may also have an important role in the transport, storage, and elimination of ammonia from the body.

Glycine may be used to form porphyrins, which commonly are used in the transport of oxygen. Glycine, aspartate and glutamine may be used in the synthesis of purine and pyrimidine bases for use in nucleotides and management of genetic material. Arginine and glycine are important components in the synthesis of creatine (also an amino acid) which is important for supplying energy for muscle function. Tryptophan, tyrosine, and histidine may be used to form many important neurotransmitters (e.g., serotonin, melatonin, catecholamines, dopamine, and histamine).

A number of other amino acids that may have important homeostasis and physiological functions in animals include, for example and not by way of limitation, homocysteine, homoserine, carnitine, creatine, ornithine, citrulline, arginosuccinic acid, 3,4-dihydroxyphenylalanine (DOPA), gamma-aminobutyric acid (also referred to as gamma-aminobutyrate; GABA), glutathione, taurine, and thyroxine as well as many others. Ornithine may play an important role in the biosynthesis of polyamines, which may be critical to DNA synthesis and cell replication. Through tests conducted by the inventors of the present invention, they have demonstrated that ornithine and GABA occur in fenugreek. Trimethylhistidine is a quaternary ammonium compound that has a structure similar to the amino acid histidine and may also be found in fenugreek.

Humans and other mammals who are subjected to (or subject themselves to) athletic performance (e.g., sports, body training, body building) may desire compositions and methods to promote muscle function (i.e., contraction, relaxation, growth) or to promote more lean body mass and fat burning. A number of muscle growth supplement compounds may be used to promote muscle growth. Such muscle growth supplement compounds may include branched chain amino acids, growth hormone, whey protein, casein protein, creatine, glutamine, androstenedione, and/or dehydroepiandrosterone (DHEA). It may be advantageous to combine one or more of these muscle growth supplements with novel compositions and methods of bio-active compounds derived, isolated, and/or extracted from fenugreek seeds. Moreover, those who may be suffering from one or more of the conditions previously described, may also have difficulty with glycogen storage and may desire compositions and methods to rapidly promote glycogen synthesis in muscle, liver, and other areas of the body.

Clinical studies conducted by the inventors of the present invention indicate that novel compositions of bio-active compounds derived, isolated, and/or extracted from fenugreek seeds, which compositions may contain 4-hydroxyisoleucine and one or more amino acids at certain concentrations, effectively enhance the transport of glucose into skeletal muscle cells in response to the presence of glucose transport factor 4 (GT-4) on skeletal muscle cells. It has been well established that the behavior of cells relative to GT-4 is very strongly correlated to the behavior of a cell relative to insulin. Therefore, a supportable indication that novel compositions of bio-active compounds derived, isolated, and/or extracted from fenugreek seeds may be used to enhance glucose transport into skeletal muscles of humans and ultimately promote glycogen synthesis is a primary focus of the present invention.

The present invention contemplates novel compositions and methods for promoting the synthesis of glycogen. As is well known in the art, glycogen is a carbohydrate and is important to any number of biochemical processes occurring in mammals.

Presently preferred embodiments of novel compositions of the present invention for promoting glycogen synthesis may include 4-hydroxyisoleucine. Preferably, the compositions of the present invention includes bio-active compounds derived, isolated, and/or extracted from the seeds of Trigonella foenum graecum, which is a botanical name for fenugreek.

One presently preferred embodiment of a composition of the present invention for promoting glycogen synthesis may include, in addition to 4-hydroxyisoleucine, one or more amino acids selected from the group consisting of: arginine, aspartate, threonine, serine, glutamate, proline, glycine, alanine, cysteine, valine, methionine, isoleucine, leucine, tryptophan, phenylalanine, ornithine, lysine, histidine, gamma-aminobutyrate, and tyrosine. In an alternate presently preferred embodiment of the present invention, a composition of bio-active compounds derived, isolated, and/or extracted from fenugreek seeds for promoting glycogen synthesis may include 4-hydroxyisoleucine and the following amino acids: arginine, aspartate, threonine, serine, glutamate, glycine, alanine, cysteine, valine, methionine, isoleucine, leucine, phenylalanine, lysine, and histidine.

One presently preferred embodiment of a composition for promoting glycogen synthesis of the present invention may comprise between about ten percent (10%) and about ninety percent (90%) amino acids and, more particularly, may comprise between about ten percent (10%) and about seventy percent (70%) 4-hydroxyisoleucine with between about twenty percent (20%) and about forty percent (40%) amino acids. In addition, a carbohydrate may be combined with the novel composition of bio-active compounds for promoting glycogen synthesis. For example, another presently preferred embodiment of the composition of the present invention may include: (1) 4-hydroxyisoleucine; (2) one or more of the following amino acids selected from the group consisting of arginine, aspartate, threonine, serine, glutamate, proline, glycine, alanine, cysteine, valine, methionine, isoleucine, leucine, tryptophan, phenylalanine, ornithine, lysine, histidine, gamma-aminobutyrate, and tyrosine; and (3) a carbohydrate selected from the group consisting of galactose, fructose, mannose, ribose, ribulose, arabinose, xylose, lyxose, and xylulose.

One presently preferred process, technique, or method of the present invention for promoting glycogen synthesis may include the step of administering an effective amount of 4-hydroxyisoleucine. An effective amount of arginine, aspartate, threonine, serine, glutamate, glycine, alanine, cysteine, valine, methionine, isoleucine, leucine, phenylalanine, lysine, and histidine may also be administered in combination with the effective amount of 4-hydroxyisoleucine to promote glycogen synthesis. The amount of 4-hydroxyisoleucine and amino acids administered for promoting glycogen synthesis are preferably derived, isolated, and/or extracted from fenugreek seeds.

As further contemplated herein, one presently preferred method of the present invention for promoting glycogen synthesis may further comprise the step of stimulating glucose transport proteins to translocate glucose into liver, muscle, kidney, and intestine. The glucose transport proteins may include glucose transport protein types 1-7 (GT 1-7), and preferably glucose transport protein type 4 (GT 4).

In addition, one presently preferred embodiment of a method for promoting glycogen synthesis of the present invention may comprise the additional steps of increasing serum insulin concentration within from about one (1) minute to about seventy-five (75) minutes following the initial administration of a novel composition of bio-active compounds derived, isolated, and/or extracted from fenugreek seeds. Preferably, the step of increasing serum insulin concentration following administration of a composition of the present invention contemplates between from about ten (10) minutes to about thirty (30) minutes and, more particularly, within about fifteen (15) minutes following administration.

The routes of administration for delivering novel compositions of the present invention for promoting glycogen synthesis may include oral, parenteral, sublingual, topical, transdermal, intramuscular, intranasal, and inhalation. Dosage delivery forms may be from any pharmaceutical, nutraceutical, and/or functional food form and may include, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Moreover, an effective amount of a novel composition of the present invention for promoting glycogen synthesis may include between about one (1) and about nine (9) mg 4-hydroxyisoleucine per kg body weight, and preferably about two (2) mg 4-hydroxyisoleucine per kg body weight.

One presently preferred embodiment of a composition of the present invention for promoting glycogen synthesis may include an effective amount of 4-hydroxyisoleucine in combination with one or more of the following compounds: amino acid, protein, nucleotide, vitamin, mineral and/or electrolyte, carbohydrate, herbal and/or botanical, and the like. Amino acids may include, for example and not by way of limitation, arginine, aspartate, threonine, serine, glutamate, glycine, alanine, cysteine, valine, methionine, isoleucine, leucine, phenylalanine, lysine, histidine, proline, tryptophan, ornithine, gamma-aminobutyrate, and tyrosine. Proteins may include, for example and not by way of limitation, growth hormone, whey protein, and casein protein. Nucleotide may include, for example and not by way of limitation, adenosine triphosphate (ATP), adenosine diphosphate (ADP), adenosine monophosphate (AMP), cyclic adenosine monophosphate (cAMP), guanosine triphosphate (GTP), guanosine diphosphate (GDP), nicotinamide adenine dinucleotide (NAD), nicotinamide adenine dinucleotide phosphate (NADP), flavin adenine dinucleotide (FAD), and the like. Vitamin may include, for example and not by way of limitation, a fat-soluble vitamin (e.g., vitamin A, vitamin D, vitamin E, vitamin K), a B-complex vitamin (e.g., vitamin B1 (thiamine), vitamin B2 (riboflavin), vitamin B3 (niacin), vitamin B5 (pantothenic acid), vitamin B6 (pyridoxine), vitamin B12 (cyanocobalamin), folic acid (sometimes referred to as vitamin B9)), vitamin C, and vitamin co-factors (e.g., biotin), and the like. Mineral and/or electrolyte may include, for example and not by way of limitation, sodium, magnesium, phosphorous, potassium, calcium, vanadium, chromium, manganese, iron, zinc, selenium, and the like. Carbohydrate may include, for example and not by way of limitation, glucose, glycogen, saccharide, sugar, and the like. Herbal and/or botanical may include, for example and not by way of limitation, ginkgo, ginseng, green tea extract, Tribulus Terrestris extract, White Willow extract, and the like.

The effective amount of 4-hydroxyisoleucine and other amino acids of the presently preferred embodiments of the novel composition of bio-active compounds for promoting glycogen synthesis may be derived, isolated, and/or extracted from fenugreek seeds.

The present invention further contemplates novel compositions and methods for promoting muscle function. As the term is used herein, “muscle function” includes a complex physiological mechanism which may involve muscle contraction, relaxation, and growth. One presently preferred embodiment of novel compositions of the present invention for promoting muscle function may include a combination of 4-hydroxyisoleucine, arginine, aspartate, threonine, serine, glutamate, glycine, alanine, cysteine, valine, methionine, isoleucine, leucine, phenylalanine, lysine, and histidine. One or more of the following amino acids may also be included: proline, tryptophan, ornithine, gamma-aminobutyrate, and tyrosine. Preferably, the compositions of the present invention include bio-active compounds derived, isolated, and/or extracted from fenugreek seeds. Moreover, an effective amount of a novel composition of the present invention for promoting muscle function may include between about one (1) and about nine (9) mg 4-hydroxyisoleucine per kg body weight, and preferably about two (2) mg 4-hydroxyisoleucine per kg body weight.

One presently preferred embodiment of a composition for promoting muscle function of the present invention may comprise between about ten percent (10%) and about ninety percent (90%) amino acids and, more particularly, may comprise between about ten percent (10%) and about seventy percent (70%) 4-hydroxyisoleucine with between about twenty percent (20%) and about forty percent (40%) amino acids. In addition, a carbohydrate may be combined with the novel composition of bio-active compounds for promoting muscle function. The carbohydrate may include at least one sugar selected from the group consisting of glucose, galactose, fructose, mannose, ribose, ribulose, arabinose, xylose, lyxose, and xylulose. It will be appreciated that the foregoing list of sugars is intended to be exemplary and not exhaustive.

In yet another presently preferred embodiment of the present invention, a composition of bio-active compounds derived, isolated, and/or extracted from fenugreek seeds for promoting muscle function may be further combined with one or more muscle growth supplements. For example, a composition for promoting muscle function may include: (1) 4-hydroxyisoleucine; (2) arginine, aspartate, threonine, serine, glutamate, glycine, alanine, cysteine, valine, methionine, isoleucine, leucine, phenylalanine, lysine, and histidine; (2) one or more of the following amino acids: proline, tryptophan, ornithine, gamma-aminobutyrate, and tyrosine; and (3) one or more of the following muscle growth supplements: branched chain amino acids, arginine, lysine, methionine, histidine, growth hormone, whey protein, creatine, glutamine, adrostenedione, and dehydroepiandrosterone (DHEA).

One presently preferred process, technique, or method of the present invention for promoting muscle function may include the step of administering an effective amount of 4-hydroxyisoleucine. An effective amount of arginine, aspartate, threonine, serine, glutamate, glycine, alanine, cysteine, valine, methionine, isoleucine, leucine, phenylalanine, lysine, and histidine may also be administered in combination with the effective amount of 4-hydroxyisoleucine to promote muscle function. In one presently preferred embodiment of the present invention, the amount of 4-hydroxyisoleucine and amino acids administered are preferably derived, isolated, and/or extracted from fenugreek seeds.

As further contemplated herein, one presently preferred method of the present invention for promoting muscle function may further comprise the step of storing energy by stimulating glucose transport proteins to translocate glucose into muscle. The glucose transport proteins may include glucose transport protein types 1-7 (GT 1-7), and preferably glucose transport protein type 4 (GT 4).

In addition, one presently preferred embodiment of a method for promoting muscle function of the present invention may comprise the additional steps of administering an effective amount of 4-hydroxyisoleucine, arginine, aspartate, threonine, serine, glutamate, glycine, alanine, cysteine, valine, methionine, isoleucine, leucine, phenylalanine, lysine, and histidine in combination with one or more muscle growth supplements. Muscle growth supplements may include one or more selected from the following group: branched chain amino acids, arginine, lysine, methionine, histidine, growth hormone, whey protein, casein protein, creatine, glutamine, androstenedione, and dehydroepiandrosterone (DHEA). It will be appreciated that the foregoing list of muscle growth supplements is intended to be exemplary and not exhaustive.

A presently preferred method for promoting muscle function may include the step of administering a carbohydrate in combination with the novel composition of bio-active compounds derived, isolated, and/or extracted from fenugreek seeds. The carbohydrate may include at least one sugar selected from the group consisting of glucose, galactose, fructose, mannose, ribose, ribulose, arabinose, xylose, lyxose, and xylulose.

The routes of administration for delivering novel compositions of the present invention for promoting muscle function may include oral, parenteral, sublingual, topical, transdermal, intramuscular, intranasal, and inhalation. Dosage delivery forms may be from any pharmaceutical, nutraceutical, and/or functional food form and may include, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Another presently preferred embodiment of the present invention contemplates novel compositions and methods for promoting muscle function in mammals. Examples of enhancing muscle function, may include but are not limited to, improvement in muscle contraction, relaxation, and/or growth. Muscle contraction may include improvements in the shortening and/or thickening of a muscle or muscle fiber in response to a voluntary or involuntary stimulus. Muscle relaxation improvements may include the lengthening of muscles or muscle fiber that occurs upon muscle deactivation. Muscle growth improvements may include an increase in the size and/or dimension of muscle or muscle fiber.

One presently preferred embodiment of a composition of the present invention for promoting muscle function may include an effective amount of 4-hydroxyisoleucine in combination with one or more of the following compounds: amino acid, protein, nucleotide, vitamin, mineral and/or electrolyte, carbohydrate, herbal and/or botanical, and the like. Amino acids may include, for example and not by way of limitation, arginine, aspartate, threonine, serine, glutamate, glycine, alanine, cysteine, valine, methionine, isoleucine, leucine, phenylalanine, lysine, histidine, proline, tryptophan, ornithine, gamma-aminobutyrate, and tyrosine. Proteins may include, for example and not by way of limitation, growth hormone, whey protein, and casein protein. Nucleotide may include, for example and not by way of limitation, adenosine triphosphate (ATP), adenosine diphosphate (ADP), adenosine monophosphate (AMP), cyclic adenosine monophosphate (cAMP), guanosine triphosphate (GTP), guanosine diphosphate (GDP), nicotinamide adenine dinucleotide (NAD), nicotinamide adenine dinucleotide phosphate (NADP), flavin adenine dinucleotide (FAD), and the like. Vitamin may include, for example and not by way of limitation, a fat-soluble vitamin (e.g., vitamin A, vitamin D, vitamin E, vitamin K), a B-complex vitamin (e.g., vitamin B1 (thiamine), vitamin B2 (riboflavin), vitamin B3 (niacin), vitamin B5 (pantothenic acid), vitamin B6 (pyridoxine), vitamin B12 (cyanocobalamin), folic acid (sometimes referred to as vitamin B9)), vitamin C, and vitamin co-factors (e.g., biotin), and the like. Mineral and/or electrolyte may include, for example and not by way of limitation, sodium, magnesium, phosphorous, potassium, calcium, vanadium, chromium, manganese, iron, zinc, selenium, and the like. Carbohydrate may include, for example and not by way of limitation, glucose, glycogen, saccharide, sugar, and the like. Herbal and/or botanical may include, for example and not by way of limitation, ginkgo, ginseng, green tea extract, Tribulus Terrestris extract, White Willow extract, and the like.

The effective amount of 4-hydroxyisoleucine and other amino acids of the presently preferred embodiments of the novel composition of bio-active compounds for promoting muscle function may be derived, isolated, and/or extracted from fenugreek seeds.

Another presently preferred embodiment of the present invention contemplates novel compositions and methods for enhancing glucose disposal in mammals. “Glucose disposal,” as used herein, refers to the removal of glucose from one area to another area of the body (i.e., transfer of glucose from one place to another). Examples of enhancing glucose disposal, may include but are not limited to, increasing absorption of glucose from intestine, increasing absorption of glucose from blood or plasma into tissues (e.g., muscle), and stimulating pancreatic beta cells to produce and secrete insulin. Enhancing glucose disposal may be graphically depicted by a decrease in the total area under the curve. Total area under the curve may be a plot of glucose concentration in one area of the body over a measured time period.

One presently preferred embodiment of a composition of the present invention for enhancing glucose disposal may include an effective amount of 4-hydroxyisoleucine, arginine, aspartate, threonine, serine, glutamate, glycine, alanine, cysteine, valine, methionine, isoleucine, leucine, phenylalanine, lysine, and histidine. In addition, a composition for enhancing glucose disposal may further include an effective amount of one or more amino acids selected from the group consisting of proline, tryptophan, ornithine, gamma-aminobutyrate, and tyrosine. The effective amount of 4-hydroxyisoleucine and other amino acids of the presently preferred embodiments of the novel composition of bio-active compounds for enhancing glucose disposal may be derived, isolated, and/or extracted from fenugreek seeds.

In one presently preferred embodiment, a composition ob bio-active compounds for enhancing glucose disposal may comprise a range between ten percent (10%) and ninety percent (90%) of amino acids and more particularly, may comprise a range between ten percent (10%) and seventy percent (70%) 4-hydroxyisoleucine with a range between twenty percent (20%) and forty percent (40%) amino acids. In addition, a carbohydrate may be combined with the novel composition of bio-active compounds for enhancing glucose disposal. Preferably, the carbohydrate includes at least one sugar selected from the group consisting of glucose, galactose, fructose, mannose, ribose, ribulose, arabinose, xylose, lyxose, and xylulose.

One or more insulin response modifiers selected from the group consisting of insulin, insulin secretagogue, biguanide, alpha-glucosidase inhibitor, and thiazolidinediones may be included in a presently preferred embodiment of the compositions of bio-active compounds for enhancing glucose disposal. Preferably, the insulin secretagogue includes a sulfonylurea compound selected from the group consisting of tolbutamide, acetohexamide, tolazamide, chlorpropamide, glyburide, glipizide, glimepiride and analogues, isomers, and pharmacological salts thereof. Biguanide may further comprise metformin and analogues, isomers, and pharmacological salts thereof and the alpha-glucosidase inhibitor may include acarbose and miglitol and analogues, isomers, and pharmacological salts thereof.

One presently preferred process, technique, or method of the present invention for enhancing glucose disposal may include the steps of administering an effective amount of the presently preferred embodiments of the composition of bio-active compounds for enhancing glucose disposal described hereinabove which includes an effective amount of 4-hydroxyisoleucine. An effective amount of arginine, aspartate, threonine, serine, glutamate, glycine, alanine, cysteine, valine, methionine, isoleucine, leucine, phenylalanine, lysine, and histidine may also be administered in combination with the effective amount of 4-hydroxyisoleucine. Preferably, the amount of 4-hydroxyisoleucine and amino acids administered for enhancing glucose disposal are preferably derived, isolated, and/or extracted from fenugreek seeds.

The routes of administration for delivering novel compositions of the present invention for enhancing glucose disposal may include oral, parenteral, sublingual, topical, transdermal, intramuscular, intranasal, and inhalation. Dosage delivery forms may be from any pharmaceutical, nutraceutical, and/or functional food form and may include, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Moreover, an effective amount of a novel composition of the present invention for enhancing glucose disposal may include between about one (1) and about nine (9) mg 4-hydroxyisoleucine per kg body weight, and preferably about two (2) mg 4-hydroxyisoleucine per kg body weight.

One presently preferred embodiment of a composition of the present invention for enhancing glucose disposal may include an effective amount of 4-hydroxyisoleucine in combination with one or more of the following compounds: amino acid, protein, nucleotide, vitamin, mineral and/or electrolyte, carbohydrate, herbal and/or botanical, and the like. Amino acids may include, for example and not by way of limitation, arginine, aspartate, threonine, serine, glutamate, glycine, alanine, cysteine, valine, methionine, isoleucine, leucine, phenylalanine, lysine, histidine, proline, tryptophan, ornithine, gamma-aminobutyrate, and tyrosine. Proteins may include, for example and not by way of limitation, growth hormone, whey protein, and casein protein. Nucleotide may include, for example and not by way of limitation, adenosine triphosphate (ATP), adenosine diphosphate (ADP), adenosine monophosphate (AMP), cyclic adenosine monophosphate (cAMP), guanosine triphosphate (GTP), guanosine diphosphate (GDP), nicotinamide adenine dinucleotide (NAD), nicotinamide adenine dinucleotide phosphate (NADP), flavin adenine dinucleotide (FAD), and the like. Vitamin may include, for example and not by way of limitation, a fat-soluble vitamin (e.g., vitamin A, vitamin D, vitamin E, vitamin K), a B-complex vitamin (e.g., vitamin B1 (thiamine), vitamin B2 (riboflavin), vitamin B3 (niacin), vitamin B5 (pantothenic acid), vitamin B6 (pyridoxine), vitamin B12 (cyanocobalamin), folic acid (sometimes referred to as vitamin B9)), vitamin C, and vitamin co-factors (e.g., biotin), and the like. Mineral and/or electrolyte may include, for example and not by way of limitation, sodium, magnesium, phosphorous, potassium, calcium, vanadium, chromium, manganese, iron, zinc, selenium, and the like. Carbohydrate may include, for example and not by way of limitation, glucose, glycogen, saccharide, sugar, and the like. Herbal and/or botanical may include, for example and not by way of limitation, ginkgo, ginseng, green tea extract, Tribulus Terrestris extract, White Willow extract, and the like.

The present invention further contemplates and teaches novel compositions and methods for stimulating pancreatic beta cells to produce insulin which may include the step of administering an effective amount of 4-hydroxyisoleucine, arginine, aspartate, threonine, serine, glutamate, glycine, alanine, cysteine, valine, methionine, isoleucine, leucine, phenylalanine, lysine, and histidine. In addition, a composition for enhancing glucose disposal may further include an effective amount of one or more amino acids selected from the group consisting of proline, tryptophan, ornithine, gamma-aminobutyrate, and tyrosine. The effective amount of 4-hydroxyisoleucine and other amino acids of the presently preferred embodiments of the novel composition of bio-active compounds for stimulating pancreatic beta cells to produce insulin may be derived, isolated, and/or extracted from fenugreek seeds.

As further contemplated herein, one presently preferred method of the present invention for stimulating pancreatic beta cells to produce insulin may further comprise the step of increasing serum insulin concentration within from about one (1) minute to about seventy-five (75) minutes following the initial administration of a novel composition of bio-active compounds. Preferably, the step of increasing serum insulin concentration following administration of a composition for stimulating pancreatic beta cells of the present invention contemplates between from about ten (10) minutes to about thirty (30) minutes and, more particularly, within about fifteen (15) minutes following administration.

The routes of administration for delivering novel compositions of the present invention for stimulating pancreatic beta cells to produce insulin may include oral, parenteral, sublingual, topical, transdermal, intramuscular, intranasal, and inhalation. Dosage delivery forms may be from any pharmaceutical, nutraceutical, and/or functional food form and may include, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Moreover, an effective amount of a novel composition of the present invention for stimulating pancreatic beta cells to produce insulin may include between about one (1) and about nine (9) mg 4-hydroxyisoleucine per kg body weight, and preferably about two (2) mg 4-hydroxyisoleucine per kg body weight.

One or more insulin response modifiers selected from the group consisting of insulin, insulin secretagogue, biguanide, alpha-glucosidase inhibitor, and thiazolidinediones may be included in a presently preferred embodiment of a method for stimulating pancreatic beta cells to produce insulin by administration of a novel composition of bio-active compounds as taught herein. Preferably, the insulin secretagogue includes a sulfonylurea compound selected from the group consisting of tolbutamide, acetohexamide, tolazamide, chlorpropamide, glyburide, glipizide, glimepiride and analogues, isomers, and pharmacological salts thereof. Biguanide may further comprise metformin and analogues, isomers, and pharmacological salts thereof and the alpha-glucosidase inhibitor may include acarbose and miglitol and analogues, isomers, and pharmacological salts thereof.

One presently preferred embodiment of a composition of the present invention for stimulating pancreatic beta cells to produce insulin may include an effective amount of 4-hydroxyisoleucine in combination with one or more of the following compounds: amino acid, protein, nucleotide, vitamin, mineral and/or electrolyte, carbohydrate, herbal and/or botanical, and the like. Amino acids may include, for example and not by way of limitation, arginine, aspartate, threonine, serine, glutamate, glycine, alanine, cysteine, valine, methionine, isoleucine, leucine, phenylalanine, lysine, histidine, proline, tryptophan, ornithine, gamma-aminobutyrate, and tyrosine. Proteins may include, for example and not by way of limitation, growth hormone, whey protein, and casein protein. Nucleotide may include, for example and not by way of limitation, adenosine triphosphate (ATP), adenosine diphosphate (ADP), adenosine monophosphate (AMP), cyclic adenosine monophosphate (cAMP), guanosine triphosphate (GTP), guanosine diphosphate (GDP), nicotinamide adenine dinucleotide (NAD), nicotinamide adenine dinucleotide phosphate (NADP), flavin adenine dinucleotide (FAD), and the like. Vitamin may include, for example and not by way of limitation, a fat-soluble vitamin (e.g., vitamin A, vitamin D, vitamin E, vitamin K), a B-complex vitamin (e.g., vitamin B1 (thiamine), vitamin B2 (riboflavin), vitamin B3 (niacin), vitamin B5 (pantothenic acid), vitamin B6 (pyridoxine), vitamin B12 (cyanocobalamin), folic acid (sometimes referred to as vitamin B9)), vitamin C, and vitamin co-factors (e.g., biotin), and the like. Mineral and/or electrolyte may include, for example and not by way of limitation, sodium, magnesium, phosphorous, potassium, calcium, vanadium, chromium, manganese, iron, zinc, selenium, and the like. Carbohydrate may include, for example and not by way of limitation, glucose, glycogen, saccharide, sugar, and the like. Herbal and/or botanical may include, for example and not by way of limitation, ginkgo, ginseng, green tea extract, Tribulus Terrestris extract, White Willow extract, and the like.

The present invention further contemplates novel compositions and methods for promoting lean body mass and fat burning which may include the step of administering an effective amount of 4-hydroxyisoleucine. One presently preferred embodiment of a method for promoting lean body mass and fat burning may include the administration of a composition including 4-hydroxyisoleucine and one or more amino acids selected from the group consisting of arginine, aspartate, threonine, serine, glutamate, glycine, alanine, cysteine, valine, methionine, isoleucine, leucine, phenylalanine, lysine, and histidine. In addition, a presently preferred composition for promoting lean body mass and fat burning may include an effective amount of one or more amino acids selected from the group consisting of proline, tryptophan, ornithine, gamma-aminobutyrate, and tyrosine. The effective amount of 4-hydroxyisoleucine and other amino acids of the presently preferred embodiments of the novel composition of bio-active compounds for promoting lean body mass and fat burning are preferably derived, isolated, and/or extracted from fenugreek seeds.

As further contemplated herein, one presently preferred method of the present invention for promoting lean body mass and fat burning may further comprise the step of storing energy by stimulating glucose transport proteins to translocate glucose into muscle. The glucose transport proteins may include glucose transport protein types 1-7 (GT 1-7), and preferably glucose transport protein type 4 (GT 4).

In addition, one presently preferred embodiment of a method for promoting lean body mass and fat burning of the present invention may comprise the additional step of administering an effective amount of one or more muscle growth supplements. Muscle growth supplements may include one or more selected from the following group: branched chain amino acids, arginine, lysine, methionine, histidine, growth hormone, whey protein, casein protein, creatine, glutamine, androstenedione, and dehydroepiandrosterone (DHEA). It will be appreciated that the foregoing list of muscle growth supplements is intended to be exemplary and not exhaustive.

A presently preferred method for promoting lean body mass and fat burning may further include the step of administering a carbohydrate in combination with the novel composition of bio-active compounds. The carbohydrate may include at least one sugar selected from the group consisting of glucose, galactose, fructose, mannose, ribose, ribulose, arabinose, xylose, lyxose, and xylulose.

Yet another presently preferred embodiment of a composition of the present invention for enhancing muscle function and/or promoting lean body mass and fat burning may further include an effective amount of 4-hydroxyisoleucine in combination with one or more of the following compounds: beta adrenergic receptor agonist (e.g., epinephrine, norepinephrine, isoproterenol, dobutamine, and the like), alpha-adrenergic receptor antagonist (e.g., yohimbine, doxazosin, terazosin, prazosin, and the like), direct stimulant of adenylate cyclase (e.g., forskolin), phosphodiesterase inhibitor (e.g., caffeine, theophylline, theobromine), adenosine receptor antagonist (e.g., caffeine, and the like), metabolic cofactor (e.g., coenzyme A, alpha-lipoic acid, carnitine, pantothenic acid, lipase, and the like).

The routes of administration for delivering novel compositions of the present invention for promoting lean body mass and fat burning may include oral, parenteral, sublingual, topical, transdermal, intramuscular, intranasal, and inhalation. Dosage delivery forms may be from any pharmaceutical, nutraceutical, and/or functional food form and may include, for example and not by way of limitation, tablet, capsule, powder, granule, microgranule, pellet, soft-gel, controlled-release form, liquid, drop, lozenge, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, bar, beverage, bread, cereal, cracker, egg, juice and juice drink, milk and soft cheese, mineral water, pasta, pasta sauce, probiotic drink soya product, spread, stimulation/energy beverage, yogurt, baby and/or children's food, women's product, men's product, meal replacement, and the like.

Moreover, an effective amount of a novel composition of the present invention for promoting lean body mass and fat burning may include between about one (1) and about nine (9) mg 4-hydroxyisoleucine per kg body weight, and preferably about two (2) mg 4-hydroxyisoleucine per kg body weight.

One presently preferred embodiment of a composition of the present invention for promoting lean body mass and fat burning may include an effective amount of 4-hydroxyisoleucine in combination with one or more of the following compounds: amino acid, protein, nucleotide, vitamin, mineral and/or electrolyte, carbohydrate, herbal and/or botanical, and the like. Amino acids may include, for example and not by way of limitation, arginine, aspartate, threonine, serine, glutamate, glycine, alanine, cysteine, valine, methionine, isoleucine, leucine, phenylalanine, lysine, histidine, proline, tryptophan, ornithine, gamma-aminobutyrate, and tyrosine. Proteins may include, for example and not by way of limitation, growth hormone, whey protein, and casein protein. Nucleotide may include, for example and not by way of limitation, adenosine triphosphate (ATP), adenosine diphosphate (ADP), adenosine monophosphate (AMP), cyclic adenosine monophosphate (cAMP), guanosine triphosphate (GTP), guanosine diphosphate (GDP), nicotinamide adenine dinucleotide (NAD), nicotinamide adenine dinucleotide phosphate (NADP), flavin adenine dinucleotide (FAD), and the like. Vitamin may include, for example and not by way of limitation, a fat-soluble vitamin (e.g., vitamin A, vitamin D, vitamin E, vitamin K), a B-complex vitamin (e.g., vitamin B1 (thiamine), vitamin B2 (riboflavin), vitamin B3 (niacin), vitamin B5 (pantothenic acid), vitamin B6 (pyridoxine), vitamin B12 (cyanocobalamin), folic acid (sometimes referred to as vitamin B9)), vitamin C, and vitamin co-factors (e.g., biotin), and the like. Mineral and/or electrolyte may include, for example and not by way of limitation, sodium, magnesium, phosphorous, potassium, calcium, vanadium, chromium, manganese, iron, zinc, selenium, and the like. Carbohydrate may include, for example and not by way of limitation, glucose, glycogen, saccharide, sugar, and the like. Herbal and/or botanical may include, for example and not by way of limitation, ginkgo, ginseng, green tea extract, Tribulus Terrestris extract, White Willow extract, and the like.

In an effort to quantify the amino acid and protein content of the presently preferred embodiments of the novel compositions of the present invention disclosed hereinabove, a methods validation program may be utilized. Determination of the ratio of 4-hydroxyisoleucine and other amino acids in fenugreek may be performed using a high performance liquid chromatography (HPLC) apparatus. As contemplated herein, an HPLC apparatus including a fluorescence detector and programmable autosampler may be utilized in a methods validation program. The chromatography column may be a Zorbax stable bond SB-C18 (4.6*150 mm, 5 μm). In addition, an HPLC apparatus may include an analytical balance, accurate to 0.1 mg, an ultrasonic bath, a volumetric flask, a two liter vacuum filtration glassware with 0.2 μm membrane, variable volumetric pipets, and a magnetic stirrer and stir bars.

The reagents of a methods validation program may include, for example: (1) methanol (HPLC grade), (2) acetonitrile (HPLC grade), (3) sodium acetate trihydrate (AR grade), (4) triethylamine (AR grade), (5) glacial acetic acid (AR grade), (6) tetrafuran (AR grade), (7) OPA reagent (Agilent Co. Part No. 5061-3335, containing o-phthaldialdehyde and 3-mercaptopropionic acid in borate buffer), (8) a reference standard of 4-hydroxyisoleucine (obtained from British Agricultural Lab), and (9) de-ionized water.

One presently preferred embodiment of a methods validation program preparation may include a mobile phase step, a standard preparation step, and a sample preparation step. In the mobile phase step, buffer A, buffer B, and a filter/degas step may be utilized. Buffer A may be prepared in a one-liter beaker, wherein 1.36 g of sodium acetate trihydrate may be dissolved in 500 mL water. This combination may be stirred until thoroughly dissolved. Ninety (90) μL of triethylamine may be added and mixed. The pH may be adjusted to about 7.2 with between from about one percent (1%) to about two percent (2%) of acetic acid solution. 1.5 mL of tetrafuran may then be added and mixed. The final mixture may be labeled—“buffer A.”

Buffer B may be formed in accordance with the following procedure. In a beaker, 1.36 g sodium acetate trihydrate may be dissolved in 100 mL of water. This combination may be stirred until thoroughly dissolved. The pH may be adjusted to 7.2 with between from about one percent (1%) to about two percent (2%) acetic acid solution. 200 mL of methanol and 200 mL of acetonitrile may then be added to the beaker and mixed well. The final mixture may be labeled—“buffer B.” Preferably, the buffers may be filtered and degassed using a vacuum and 0.2 μm membrane.

In one presently preferred embodiment of the present invention, a methods validation program standard preparation step may include, accurately weighing about ten (10) mg of a reference compound and placing the compound into a fifty (50) mL volumetric flask. The reference compound may be dissolved using about thirty (30) mL deionized water and undergoing sonicate for approximately ten (10) minutes. The flask is preferably allowed to cool to room temperature and then the solution may be diluted with water to specific concentration and mixed well. The standard preparation may then be sealed with a parafilm and stored under refrigeration until needed.

A methods validation program sample preparation step may include, accurately weighing about twenty-five (25) mg of a composition of bio-active compounds extracted from fenugreek seed and dissolving with about thirty (30) mL deionized water in a fifty (50) mL volumetric flask and undergoing sonicate for approximately ten (10) minutes. The flask is preferably allowed to cool to room temperature and then the solution may be diluted with water to specific concentration and mixed well. A sample preparation may be filtered prior to being injected into an HPLC apparatus.

Chromatographic conditions for one presently preferred embodiment of a method validation program may include, for example, a Zorbax stable bond SB-C18 column, a column temperature of 30°Celsius (C.), and an EX 340 nM, EM 450 chromatographic detector. The following gradients and injection program may be utilized:

Gradient:

| Time(min) | % A | % B | F(ml/min) |
|---|---|---|---|
| 0.00 | 100 | 0 | 1.0 |
| 17.0 | 50 | 50 | 1.0 |
| 20.0 | 0 | 100 | 1.0 |
| 20.1 | 0 | 100 | 1.0 |
| 24.0 | 100 | 0 | 1.0 |
| 35.0 | 100 | 0 | 1.0 |

Injection Program:

| Row | Action |
|---|---|
| 1 | Draw 5.0 μL from vial 1 (buffer) |
| 2 | Draw 1.0 μL from vial 2 (sample) |
| 3 | Mix 6.0 μL in air, max. speed, 6 times |
| 4 | Submerge injector tip in vial 11 (wash vial) |
| 5 | Draw 1.0 μL from vial 3 (OPA reagent) |
| 6 | Mix 7.0 μL in air, max. speed, 6 times |
| 7 | Submerge injector tip in vial 11 (wash vial) |
| 8 | Inject |

A methods validation program specificity may be performed by examining the spectrum of the identified peak. This peak may show the spectra of the sample and reference standards. A methods validation program linearity may be analyzed by preparing standard preparations of 4-hydroxyisoleucine and assayed as directed in the methods validation program. One such linearity was undertaken and the following results were observed:

| Concentration | | Response |
|---|---|---|
| (mg/ml) | Peak area | (area/conc.) |
| 0.09 | 1336.9 | 3.36600e−3 |
| 0.18 | 2654.1 | 3.39098e−3 |
| 0.27 | 4040.7 | 3.34101e−3 |
| y-intercept | −26.56667 | Ave = 3.36600e−3 |
| slope | 300.4222 | SD = 0.024985 |
| correlation | 0.99989 | RSD = 0.74% |

The correlation coefficient is satisfactory R>0.99950) and these data demonstrate the methods validation program of the present invention has good linearity.

A methods validation program precision may be analyzed with six (6) separated tests performed on a test sample, if desired. One such precision analysis was undertaken and the following results were observed:

4-OH-Ile precision LOT NO: 2060052

| Number | Peak area | Assay |
|---|---|---|
| 1 | 2827.8 | 35.4 |
| 2 | 2758.2 | 35.3 |
| 3 | 2997.0 | 34.9 |
| 4 | 2721.5 | 35.1 |
| 5 | 2510.6 | 34.9 |
| 6 | 2562.2 | 35.4 |
| Average | — | 35.2 |
| SD | — | 0.234 |
| RSD | — | 0.66% |

4-OH-Ile precision LOT NO: 20020402

| Number | Peak area | Assay |
|---|---|---|
| 1 | 3369.0 | 43.6 |
| 2 | 3214.2 | 43.9 |
| 3 | 3292.9 | 43.5 |
| 4 | 3112.6 | 43.0 |
| 5 | 3394.8 | 44.1 |
| 6 | 3305.2 | 43.7 |
| average | — | 43.6 |
| SD | — | 0.378 |
| RSD | — | 0.87% |

4-OH-Ile precision LOT NO: FSE02G31-32

| Number | Peak area | Assay |
|---|---|---|
| 1 | 3762.9 | 49.2 |
| 2 | 3574.1 | 48.2 |
| 3 | 3560.3 | 48.2 |
| 4 | 3599.4 | 48.3 |
| 5 | 3629.7 | 49.1 |
| 6 | 3627.1 | 49.1 |
| average | — | 48.7 |
| SD | — | 0.496 |
| RSD | — | 1.00% |

From these results, relative standard deviation (RSD) is<three percent (3%). Based on the foregoing, the methods validation program delivered good precision for the sample.

A methods validation program was conducted and analyzed for reproducibility by testing a same sample with multiple HPLC assays on consecutive days. The following results were observed:

4-OH-Ile reproducibility LOT NO: 2060052

| Number | Day1 | Day2 | Over 2 days |
|---|---|---|---|
| 1 | 35.4 | 34.5 | |
| 2 | 35.3 | 35.7 | |
| 3 | 34.9 | 36.2 | |
| 4 | 35.1 | 34.9 | |
| 5 | 34.9 | 35.6 | |
| 6 | 35.4 | 34.6 | |
| Average | 35.2 | 35.2 | 35.2 |
| SD | 0.234 | 0.683 | 0.489 |
| RSD | 0.66% | 1.94% | 1.39% |

4-OH-Ile reproducibility LOT NO: 2002-0402

| Number | Day1 | Day2 | Over 2 days |
|---|---|---|---|
| 1 | 43.6 | 44.7 | |
| 2 | 43.9 | 43.7 | |
| 3 | 43.5 | 43.9 | |
| 4 | 43.0 | 43.5 | |
| 5 | 44.1 | 43.5 | |
| 6 | 43.7 | 45.0 | |
| Average | 43.6 | 44.0 | 43.8 |
| SD | 0.378 | 0.644 | 0.548 |
| RSD | 0.87% | 1.46% | 1.25% |

The RSD is<three percent (3%) which shows that the methods validation program has good reproducibility.

A methods validation program was conducted and analyzed for recovery and accuracy using spiked and recovered sample analyte and spiked and recovered standard analyte. The following results were observed:

4-OH-Ile Recovery

| | Sample | Spiked | Recovered | Recovery | |
|---|---|---|---|---|---|
| | Spiked | (4-OH ILE) | (4-OH ILE) | (4-OH ILE) | |
| | (mg) | (mg) | (mg) | (%) | Average (%) |
| FSE2060052 + | 10.8 | 5.34 | 5.23 | 98.0 | |
| FSE02G31 − | 22.1 | 10.92 | 10.68 | 97.8 | |
| 32 | 33.9 | 16.75 | 16.41 | 98.0 | 97.9 |
| FSE20020402 + | 10.2 | 5.04 | 4.98 | 98.7 | |
| FSE02G31 − | 21.6 | 10.67 | 10.37 | 97.2 | |
| 32 | 30.8 | 15.22 | 14.84 | 97.6 | |

These foregoing data demonstrates that the methods validation program of the present invention has good accuracy. Based on the foregoing results of the methods validation program, lots containing a composition of bio-active compounds derived, isolated, and/or extracted from fenugreek seeds of the present invention may be analyzed.

The following examples will illustrate the practice of the present invention in further detail. It will be readily understood by those skilled in the art that the following methods, formulations, and compositions of a unique, high-potency, bio-active fenugreek seed extract of the present invention, as generally described and illustrated in the Examples herein, are to be viewed as exemplary of the principles of the present invention, and not as restrictive to a particular structure or process for implementing those principles. Thus, the following more detailed description of the presently preferred embodiments of the methods, formulations, and compositions of the present invention, as represented in Examples I-VII, is not intended to limit the scope of the invention, as claimed, but is merely representative of presently preferred embodiments of the invention.

### EXAMPLE I

Based on the foregoing description and results of the methods validation program, Lot No. 2090769 was analyzed using HPLC, as previously described, and was found to contain a presently preferred embodiment of a composition of bio-active compounds derived, isolated, and/or extracted from fenugreek seeds of the present invention resulting in the following composition:

| Measurement | % (w/w) |
|---|---|
| Protein | 42.52 |
| Oil Content | 0.20 |
| Ash | 3.19 |
| Moisture | 13.10 |
| Soluble Fiber | 2.30 |
| Insoluble Fiber | 0.90 |
| Amino Acids | |
| Arginine | 1.92 |
| Aspartate | 1.94 |
| Threonine | 0.43 |
| Serine | 0.32 |
| Glutamate | 3.23 |
| Proline | 0.41 |
| Glycine | 1.03 |
| Alanine | 1.17 |
| Cysteine | 0.08 |
| Valine | 0.25 |
| Methionine | 0.29 |
| Isoleucine | 0.26 |
| Leucine | 0.28 |
| Tryptophan | 0.14 |
| Phenylalanine | 0.73 |
| Lysine | 0.22 |
| Histidine | 0.29 |
| Tyrosine | 0.03 |
| 4-hydroxyisoleucine | 24.50 |
| Total Amino Acids | 37.79 |

As illustrated hereinabove, one presently preferred embodiment of a composition of the present invention consists of about forty-three percent (43%) protein, about 0.2% oil, about 3.19% ash, about 13.10% moisture, about 2.30% insoluble fiber, about 0.90% soluble fiber and about thirty-eight percent (38%) free amino acids, including about twenty-five percent (25%) 4-hydroxyisoleucine and various quantities of the following amino acids: arginine, aspartate, threonine, serine, glutamate, proline, glycine, alanine, cysteine, valine, methionine, isoleucine, leucine, tryptophan, phenylalanine, lysine, histidine, and tyrosine.

Since the compositions of bio-active compounds derived, isolated, and/or extracted from fenugreek seeds of the present invention are configured to include 4-hydroxyisoleucine and one or more amino acids, it will be readily appreciated that a composition of bio-active compounds isolated from fenugreek seeds may contain 4-hydroxyisoleucine with one or more various amino acids as described herein. It is intended, therefore, that the examples provided herein be viewed as exemplary of the principles of the present invention, and not as restrictive to a particular structure or method for implementing those principles.

### EXAMPLE II

Based on the foregoing description and results of the methods validation program, Lot No. 2121492 was analyzed using HPLC, as previously described, and was found to contain another presently preferred embodiment of a composition of bio-active compounds derived, isolated, and/or extracted from fenugreek seeds of the present invention resulting in the following composition:

| Measurement | % (w/w) |
|---|---|
| Protein | 52.43 |
| Oil Content | 0.07 |
| Ash | 1.59 |
| Moisture | 8.42 |
| Soluble Fiber | 1.80 |
| Insoluble Fiber | 0.20 |
| Amino Acids | |
| Arginine | 1.46 |
| Aspartate | 1.51 |
| Threonine | 0.34 |
| Serine | 0.12 |
| Glutamate | 3.05 |
| Proline | 0.37 |
| Glycine | 0.96 |
| Alanine | 1.31 |
| Cysteine | 0.07 |
| Valine | 0.35 |
| Methionine | 0.24 |
| Isoleucine | 0.23 |
| Leucine | 0.18 |
| Tryptophan | 0.02 |
| Phenylalanine | 0.33 |
| Lysine | 0.19 |
| Histidine | 0.29 |
| Tyrosine | 0.07 |
| 4-hydroxyisoleucine | 24.40 |
| Total Amino Acids | 35.49 |

As illustrated hereinabove, the presently preferred embodiment of a composition of the present invention consists of about fifty-two percent (52%) protein, about 0.07% oil, about 1.59% ash, about 8.42% moisture, about 1.80% insoluble fiber, about 0.20% soluble fiber and about thirty-five percent (35%) free amino acids, including about 24% 4-hydroxyisoleucine and various quantities of the following amino acids: arginine, aspartate, threonine, serine, glutamate, proline, glycine, alanine, cysteine, valine, methionine, isoleucine, leucine, tryptophan, phenylalanine, lysine, histidine, and tyrosine.

Since the compositions of bio-active compounds derived, isolated, and/or extracted from fenugreek seeds of the present invention are configured to include 4-hydroxyisoleucine and one or more amino acids, it will be readily appreciated that a composition of bio-active compounds isolated from fenugreek seeds may contain 4-hydroxyisoleucine with one or more various amino acids as described herein. It is intended, therefore, that the examples provided herein be viewed as exemplary of the principles of the present invention, and not as restrictive to a particular structure or method for implementing those principles.

### EXAMPLE III

Based on the foregoing description and results of the methods validation program, Lot No. 2101114 was analyzed using HPLC, as previously described, and was found to contain yet another presently preferred embodiment of a composition of bio-active compounds derived, isolated, and/or extracted from fenugreek seeds of the present invention resulting in the following composition:

| Measurement | % (w/w) |
|---|---|
| Amino Acids | |
| Arginine | 1.09 |
| Aspartate | 1.82 |
| Threonine | 0.41 |
| Serine | 1.71 |
| Glutamate | 3.09 |
| Proline | 0.20 |
| Glycine | 0.94 |
| Alanine | 1.48 |
| Cysteine | 0.79 |
| Valine | 0.46 |
| Methionine | 0.15 |
| Isoleucine | 0.21 |
| Leucine | 0.20 |
| Tryptophan | 0.81 |
| Phenylalanine | 0.73 |
| Lysine | 0.17 |
| Histidine | 0.16 |
| Ornithine | 0.06 |
| Gamma-aminobutyrate | 0.34 |
| 4-hydroxyisoleucine | 26.00 |
| Total Amino Acids | 40.82 |

As appreciated by those skilled in the art, the presently preferred embodiment of the present invention consists of about forty-one percent (41%) free amino acids, including about twenty-six percent (26%) 4-hydroxyisoleucine and various quantities of the following amino acids: arginine, aspartate, threonine, serine, glutamate, proline, glycine, alanine, cysteine, valine, methionine, isoleucine, leucine, tryptophan, phenylalanine, lysine, histidine, ornithine, and gamma-aminobutyrate.

Since the compositions of bio-active compounds derived, isolated, and/or extracted from fenugreek seeds of the present invention are configured to include 4-hydroxyisoleucine and one or more amino acids, it will be readily appreciated that a composition of bio-active compounds isolated from fenugreek seeds may contain 4-hydroxyisoleucine with one or more various amino acids as described herein. It is intended, therefore, that the examples provided herein be viewed as exemplary of the principles of the present invention, and not as restrictive to a particular structure or method for implementing those principles.

### EXAMPLE IV

Based on the foregoing description and results of the methods validation program, Lot No. 2101055 was analyzed using HPLC, as previously described, and was found to contain another presently preferred embodiment of a composition of bio-active compounds derived, isolated, and/or extracted from fenugreek seeds of the present invention resulting in the following composition:

| Measurement | % (w/w) |
|---|---|
| Amino Acids | |
| Arginine | 0.90 |
| Aspartate | 1.49 |
| Threonine | 0.35 |
| Serine | 4.44 |
| Glutamate | 2.47 |
| Glycine | 0.81 |
| Alanine | 1.22 |
| Cysteine | 0.67 |
| Valine | 0.41 |
| Methionine | 0.20 |
| Isoleucine | 0.20 |
| Leucine | 0.17 |
| Tryptophan | 0.69 |
| Phenylalanine | 0.61 |
| Lysine | 0.13 |
| Histidine | 0.14 |
| Ornithine | 0.04 |
| Gamma-aminobutyrate | 0.29 |
| 4-hydroxyisoleucine | 23.26 |
| Total Amino Acids | 38.49 |

As appreciated by those skilled in the art, the presently preferred embodiment of the composition consists of about thirty-nine percent (39%) free amino acids, including about twenty-three percent (23%) 4-hydroxyisoleucine and various quantities of the following amino acids: arginine, aspartate, threonine, serine, glutamate, glycine, alanine, cysteine, valine, methionine, isoleucine, leucine, tryptophan, phenylalanine, lysine, histidine, ornithine, and gamma-aminobutyrate.

Since the compositions of bio-active compounds derived, isolated, and/or extracted from fenugreek seeds of the present invention are configured to include 4-hydroxyisoleucine and one or more amino acids, it will be readily appreciated that a composition of bio-active compounds isolated from fenugreek seeds may contain 4-hydroxyisoleucine with one or more various amino acids as described herein. It is intended, therefore, that the examples provided herein be viewed as exemplary of the principles of the present invention, and not as restrictive to a particular structure or method for implementing those principles.

### EXAMPLE V

Based on the foregoing description and results of the methods validation program, Lot No. 2090898 was analyzed using HPLC, as previously described, and was found to contain yet another presently preferred embodiment of a composition of bio-active compounds derived, isolated, and/or extracted from fenugreek seeds of the present invention resulting in the following composition:

| Measurement | % (w/w) |
|---|---|
| Amino Acids | |
| Arginine | 0.81 |
| Aspartate | 1.27 |
| Threonine | 0.23 |
| Serine | 0.87 |
| Glutamate | 1.96 |
| Glycine | 0.67 |
| Alanine | 1.17 |
| Cysteine | 0.74 |
| Valine | 0.36 |
| Methionine | 0.10 |
| Isoleucine | 0.22 |
| Leucine | 0.21 |
| Phenylalanine | 0.56 |
| Ornithine | 0.08 |
| Lysine | 0.13 |
| Histidine | 0.10 |
| Tyrosine | 0.42 |
| 4-hydroxyisoleucine | 24.11 |
| Total Amino Acids | 34.01 |

As appreciated by those skilled in the art, the presently preferred embodiment of the composition consists of about 34% free amino acids, including about twenty-four percent (24%) 4-hydroxyisoleucine and quantities of the following amino acids: arginine, aspartate, threonine, serine, glutamate, glycine, alanine, cysteine, valine, methionine, isoleucine, leucine, phenylalanine, ornithine, lysine, histidine, and tyrosine.

Since the compositions of bio-active compounds derived, isolated, and/or extracted from fenugreek seeds of the present invention are configured to include 4-hydroxyisoleucine and one or more other amino acids, it will be readily appreciated that a composition of bio-active compounds isolated from fenugreek seeds may contain 4-hydroxyisoleucine with one or more various amino acids as described herein. It is intended, therefore, that the examples provided herein be viewed as exemplary of the principles of the present invention, and not as restrictive to a particular structure or method for implementing those principles.

### EXAMPLE VI

Generally referring now to FIGS. 1-5, tests were conducted with six (6) healthy recreationally active males involved in resistance or aerobic training (i.e., ≧three (3) days a week). Subjects were scheduled for a two (2) day trial to evaluate the effect of a composition of bio-active compounds derived, isolated, and/or extracted from fenugreek seeds on resting blood glucose and insulin values following oral ingestion of a glucose load (i.e., 1.8 g glucose/kg body weight (BW)) in combination with either placebo or a novel composition of bio-active compounds derived, isolated, and/or extracted from fenugreek seeds (i.e., based on 2.0 mg 4-hydroxyisoleucine/kg BW). All subjects received the placebo arm first in order to minimize carryover effect.

Methodology

Subjects arrived at the lab following an overnight fast (i.e., at least 12 hours). Experimental trials were separated by no less than 24 hours. An indwelling venous catheter (20 gauge) was inserted into an anticubital arm vein of each subject and kept patent using a continuous saline intravenous drip. Following the collection of a baseline blood sample, each subject received an oral glucose tolerance test beverage (i.e., 1.8 grams/kg BW) and either a placebo or a novel composition of bio-active compounds derived, isolated, and/or extracted from fenugreek seeds (i.e., based on 2.0 mg/kg 4-hydroxyisoleucine) in the form of three identical tablets.

Following the initial ingestion of the glucose tolerance test beverage and supplement, subsequent whole blood samples were collected from the subject at time zero (i.e., before feeding) and at times fifteen (15), twenty-five (25), thirty (30), forty-five (45), sixty (60), and seventy-five (75) minutes (i.e., minutes post-feeding). Whole blood samples were placed in plain tubes and centrifuged for about twenty (20) minutes at approximately 3000 rpm. The resultant serum was separated and stored at −30° C. for subsequent analysis.

Analysis of blood glucose was completed in duplicate by means of a Milton Roy spectrophotomer using a commercially available assay kit (e.g., Sigma, glucose infinity reagent). Insulin was assayed using a commercially available enzymatic kit (e.g., DRG, International—Human Insulin EIA-2935). All samples were measured in duplicate. The average coefficient of variation for the glucose assay was 2.1±1.8%. The average coefficient of variation for the insulin assay was 8.9±4.0%.

Results

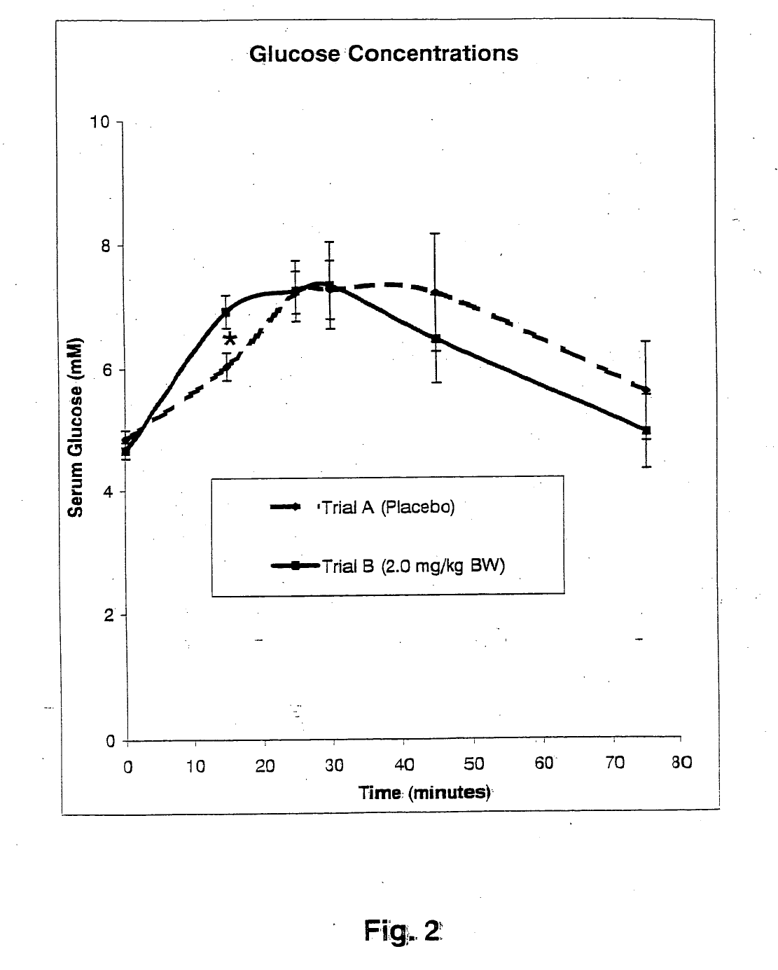

With reference to FIG. 1, a graph is shown of the insulin and glucose concentrations plotted over the time course of the post-feeding sample times. As illustrated, the vertical axis (i.e., ordinate) references insulin (μIU/mL) and glucose (mM) units and the horizontal axis (i.e., abscissa) references time (minutes). The confidence intervals for the plotted values are indicated by brackets. The top two curves illustrate the changes in glucose concentration following administration of an oral glucose bolus with placebo (dashed line) or a novel composition of bio-active compounds derived, isolated, and/or extracted from fenugreek seeds (solid line). The bottom two curves illustrate the changes in insulin concentration following administration of an oral glucose bolus with placebo (dashed line) or a novel composition of bio-active compounds derived, isolated, and/or extracted from fenugreek seeds (solid line). These data are further discussed below and in FIGS. 2-5.

Referring now to FIG. 2, a graph is shown of the serum glucose concentration plotted over the time course of the post-feeding sample times. As illustrated, the vertical axis (i.e., ordinate) references serum glucose concentrations measured in milliMolar (mM) units. The horizontal axis (i.e., abscissa) references the sample times from time zero (0) (i.e., just before receiving glucose and the experimental feeding) to time seventy-five (75) minutes. The confidence intervals for the plotted values are indicated by brackets. The data for the serum glucose response following placebo feedings are indicated by a dashed line and the data for serum glucose response following feedings with a novel composition of bio-active compounds derived, isolated, and/or extracted from fenugreek seeds are indicated by a solid line. The fifteen (15) minute time point is marked with an asterisk (i.e., *) indicates a significant difference in serum glucose concentrations between feedings with a novel composition of bio-active compounds derived, isolated, and/or extracted from fenugreek seeds and feedings with a placebo.

There were no significant differences between the trials for blood glucose at other time points. As shown, the total area under the curve (AUC) for blood glucose was similar for both trials during the initial zero (0) to forty-five (45) minute time segment. However, the total AUC was significantly lower during the experimental trial for the forty-five (45) to seventy-five (75) minute time segment and for the overall zero (0) to seventy-five (75) minute time segment.

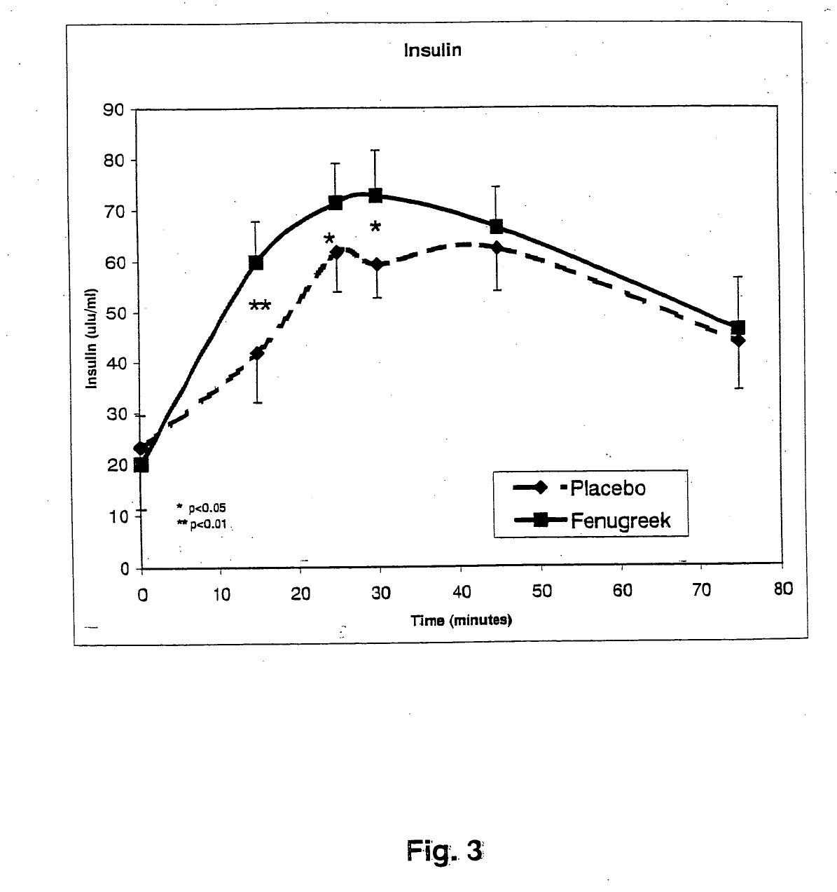

As shown in FIG. 3, a graph is shown of the insulin concentration plotted over the time course of the post-feeding sample times. As illustrated, the vertical axis (i. e., ordinate) references serum insulin activity concentrations measured in micro International Units per milliliter (μIU/mL) units. The horizontal axis (i.e., abscissa) references the sample times from time zero (0) (i.e., just before receiving glucose and the experimental feeding) to time seventy-five (75) minutes. The confidence intervals for the plotted values are indicated by brackets. The data for the serum insulin response following placebo feedings are indicated by a broken line and the data for the serum insulin response following feeding with a novel composition of bio-active compounds derived, isolated, and/or extracted from fenugreek seeds are indicated by a solid line. Time points twenty-five (25) and thirty (30) minutes are marked with an asterisk (i.e., *) indicating significant difference (i.e., p<0.05) in insulin concentrations between feedings with a novel composition of bio-active compounds derived, isolated, and/or extracted from fenugreek seeds and feedings with placebo. Time point fifteen (15) minutes is marked with a double asterisk (i.e., **) indicating highly significant difference (i.e., p<0.0 1) in insulin concentrations between feeding with a novel composition of bio-active compounds derived, isolated, and/or extracted from fenugreek seeds and feeding with a placebo. These significant differences indicate a more abrupt increase in insulin concentration with the administration of a novel composition of bio-active compounds derived, isolated, and/or extracted from fenugreek seeds.

As illustrated in FIG. 3, the serum insulin concentrations were similar across trials for the forty-five (45) and seventy-five (75) minute time points. The total area under the curve was significantly higher for the experimental trial for a number of time segments.

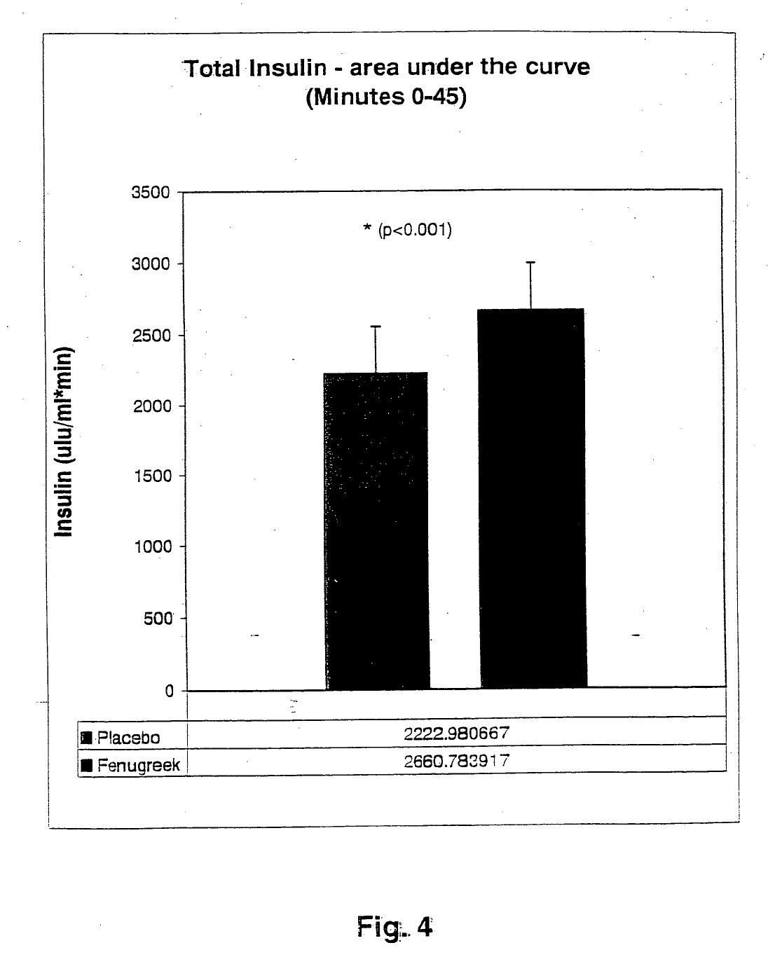

Referring now to FIG. 4, a bar graph illustrates the area under the curve for total insulin for time zero (0) to forty-five (45) minutes. The vertical axis (i.e., ordinate) referencing the insulin concentration, is measured in micro International Units per milliliter per minute (μIU/mL/min). The left bar illustrates the total area under the curve for the placebo trial and the right bar illustrates the total area under the curve for a trial with a novel composition of bio-active compounds derived, isolated, and/or extracted from fenugreek seeds. As further illustrated FIG. 4, there is a most notable indication of the differences during the early stages of the trial which coincide with a rapid release of insulin during the initial thirty (30) minutes post glucose ingestion.

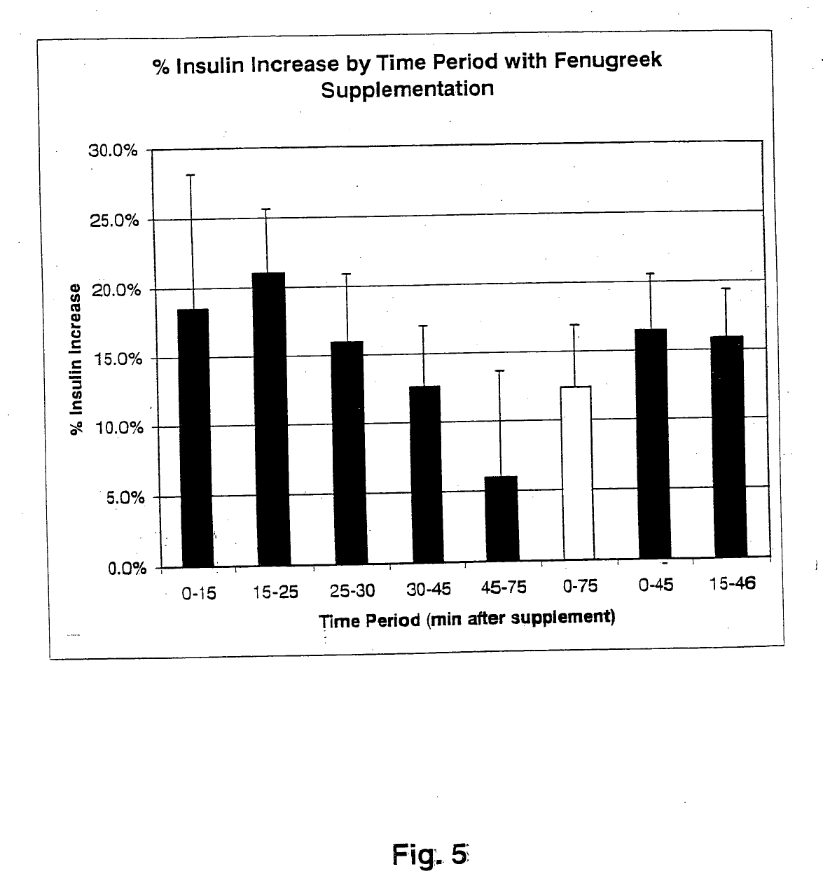

As shown in FIG. 5, a bar chart illustrates the percent (%) insulin concentration increase by time period following administration of a novel composition of bio-active compounds derived, isolated, and/or extracted from fenugreek seeds. The vertical axis (i.e., ordinate) references percent (%) insulin increase and the horizontal axis (i.e., abscissa) references time periods (measured in minutes following feeding with a presently preferred embodiment of a novel composition of bio-active compounds derived, isolated, and/or extracted from fenugreek seeds. The overall total area under the curve (i.e., zero (0) to seventy-five (75) minutes; as indicated by the hollow bar) was significantly higher for the experimental trial compared to placebo. As shown by the first three bars in FIG. 5 (as read from the left side), the experimental trial resulted in an insulin release pattern between from about fifteen percent (15%) to about twenty-one percent (21%) higher during the initial thirty (30) minutes following oral glucose ingestion.

Discussion

This example demonstrates the effects of an oral preparation containing a composition which includes the active amino acid potentiator, 4-hydroxyisoleucine, in promoting insulin release. The main findings indicate that, in combination with a large oral bolus of glucose (i.e., 1.8 g/kg BW), a novel composition of bio-active compounds derived, isolated, and/or extracted from fenugreek seeds (based on 2.0 mg 4-hydroxyisoleucine/kg) resulted in an abrupt and significantly greater insulin release during the initial thirty (30) minutes following administration. Moreover, the total area under the curve (i.e., from about time zero (0) to about seventy-five (75) minutes) for serum insulin was significantly higher for a novel composition of bio-active compounds derived, isolated, and/or extracted from fenugreek seeds compared to placebo.

Serum insulin concentration was increased during the experimental trial by an average of 18.5% during the initial fifteen (15) minutes following the oral glucose bolus compared to the placebo. The insulin concentration was 14.2% higher on average during the experimental trial for the entire time zero (0) to seventy-five (75) minute post-glucose ingestion.

In summary, the novel composition of bio-active compounds isolated from fenugreek seeds of one presently preferred embodiment of the present invention appears to alter the physiological responses associated with a large oral bolus of glucose (i.e., 1.8 g/kg BW). These physiological responses may include, but are not limited to, the following: (1) an increase in gut absorption of glucose, as evidenced by the higher blood glucose at time fifteen (15) minutes during the experimental trial; (2) a stimulation of pancreatic beta cells (as evidenced by the rapid and sustained insulin concentrations during the experimental trial); and (3) enhanced glucose disposal (i.e., transference of something into a new place; e.g., as evidenced by the smaller total area under the curve for glucose in the experimental trial, indicating glucose is being transferred out of the blood and into tissues).

Since the compositions of bio-active compounds derived, isolated, and/or extracted from fenugreek seeds of the present invention are configured to support metabolism and the transportation of glucose and other carbohydrates in animals and humans, it will be readily appreciated that a composition of bio-active compounds isolated from fenugreek seeds may contain 4-hydroxyisoleucine and one or more various bio-active compounds as described herein. Moreover, novel compositions of bio-active compounds from fenugreek seeds of the present invention may promote any number of physiological responses to administration of glucose or other carbohydrates, for example, but not by way of limitation, increased gut absorption of glucose, stimulation of pancreatic beta cells, enhanced glucose disposal from the blood. It is intended, therefore, that the examples provided herein be viewed as exemplary of the principles of the present invention, and not as restrictive to a particular structure or method for implementing those principles.

### EXAMPLE VII

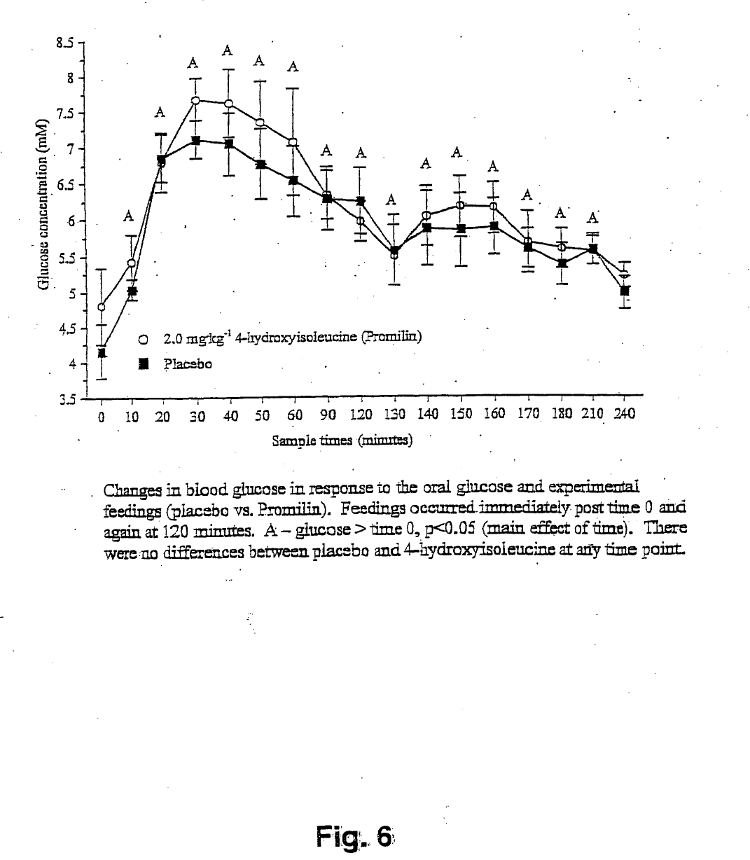

Generally referring now to FIGS. 6-8, tests were conducted with six (6) healthy human male cyclists. Following preliminary tests for peak oxygen consumption (cycle ergometer) and body composition (hydrodensitometry), the subjects were scheduled for two (2) glycogen depletion rides followed by a re-feeding protocol. The placebo and experimental trials were completed using a randomized, double-blind crossover design.

Methodology and Glycogen Depletion/Re-Feeding Trials

Following an overnight fast (i.e., at least 12 hours), the subjects reported to the lab at about 6:00 A.M. After the necessary adjustments were made to the cycle ergometer, the subjects completed a ten (10) minute warm up at approximately fifty-five percent (55%) peak VO₂ (oxygen consumption). Thereafter, the subjects completed a series often (10) intervals which included two (2) minutes at approximately eighty percent (80%) peak VO₂ followed by four (4) minutes at approximately fifty percent (50%) peak VO₂. After the series of ten (10) intervals, the subjects completed eight (8) minutes at sixty percent (60%) peak VO₂, followed by twelve (12) minutes at fifty percent (50%) peak VO₂.

Immediately upon completion of the 90-minute glycogen depletion ride, the subjects were instructed to towel off and change into dry clothes. With a subject in the supine position, an indwelling venous catheter (20 gauge) was inserted into an arm vein and kept patent using a continuous saline intravenous drip. The vastus lateralis of each of the subjects was prepared and a muscle biopsy was obtained from the vastus lateralis muscle. Following the collection of the biopsy, the incision was closed using steri-strips and the limb was slightly wrapped with an ace bandage. The initial post exercise biopsy was collected approximately ten (10)-minutes post exercise.

Following the biopsy and the collection of a baseline blood sample, each subject received an oral dose of dextrose (i.e., 1.8 grams/kg body weight, Fisher Scientific; glucose tolerance test beverage) and either a placebo or a novel composition of bio-active compounds derived, isolated, and/or extracted from fenugreek seeds (i.e., based on 2.0 mg 4-hydroxyisoleucine/kg). Following the initial ingestion of the glucose tolerance test beverage and composition of bio-active compounds derived, isolated, and/or extracted from fenugreek seeds, subsequent blood samples were obtained every ten (10) minutes until sixty (60) minutes and every thirty (30) minutes thereafter until 120 minutes. At 120 minutes post-feeding, a second oral dextrose solution (i.e., 1.8 grams/kg body weight) with either supplement (i.e., 2.0 mg/kg 4-hydroxyisoleucine) or placebo was ingested. Subsequent blood samples were obtained at ten (10) minute intervals until 180 minutes and every thirty (30) minutes thereafter until 240 minutes had expired. During the four (4) hour post-exercise session, the subjects remained in a supine and/or partially seated position in a hospital bed and were allowed to study, read, and/or watch television.

At approximately four (4) hours following the initial feeding, a second biopsy was obtained from a second incision in the vastus laterals muscle from each subject. The biopsy was obtained approximately four (4) centimeters proximal to the initial biopsy on the same leg. After excess blood and any connective tissue or fat was removed, tissue samples were immersed in liquid nitrogen within approximately one (1) minute post biopsy.

Blood samples were analyzed for glucose in duplicate using an enzymatic spectrophotometric method (e.g., Thermotrace glucose infinity reagent). Insulin was also analyzed in duplicate using an enzymatic spectrophotometric method (e.g., 96 well ELISA, DRG International).

Muscle samples were analyzed using a similar enzymatic spectrophotometric method (e.g., Thermotrace glucose infinity reagent) after tissue preparation. Briefly, samples were weighed upon removal from a −80° C. freezer. Samples were placed in a one (1) Normal HCL solution and homogenized using a manual mortar and pestle tissue grinder. Once homogenized, samples were incubated at approximately 95.6° C. for about three hours. After the incubation, one (1) Normal NaOH was added to each sample tube to normalize pH. Samples were analyzed in triplicate against known glycogen and glucose controls run at the same time. Muscle glycogen was expressed in μmol/kg/wet weight of muscle.

Glucose, insulin, and muscle glycogen were analyzed using repeated measures and analysis of variance (ANOVA) (e.g., trial×time). The difference in the overall rate of muscle glycogen resynthesis was analyzed with a 2-tailed dependent t-test. Statistical significance was established using an alpha level of p<0.05.

Results

Referring specifically to FIG. 6, a graph is shown of the glucose concentration plotted over the time course of the post-exercise sample times. As illustrated, the vertical axis (i.e., ordinate) references blood glucose concentrations measured in milliMolar (mM) units. The horizontal axis (i.e., abscissa) references the sample times from time zero (0) (i.e., just after completing the glycogen depletion exercise and just before receiving carbohydrate and the experimental feeding of the composition of the present invention) to time 240 minutes. The confidence intervals for the plotted values are indicated by brackets. The data for the blood glucose response following placebo feedings are indicated by a solid box and the data for a novel composition of bio-active compounds derived, isolated, and/or extracted from fenugreek seeds feedings are indicated by hollow circles. Those time points with an “A” indicate significant difference between the glucose level at the time point and the glucose level at time zero. The pattern of the curves are generally bi-modal and reflect the bolus administration of glucose and experimental feedings (i.e., a novel composition of bio-active compounds derived, isolated, and/or extracted from fenugreek seeds or placebo) at time zero (0) and at time 120 minutes.

The results illustrated in FIG. 6 also indicate there were no significant differences in glucose concentration between the placebo and experimental trials. However, the main effect for time indicated a significant increase in blood glucose from time zero (0) (i.e., pre-feeding, immediately post exercise) at minutes twenty (20) and every time point thereafter until 240 minutes. By 240 minutes post initial feeding, blood glucose had returned to pre-feeding values.

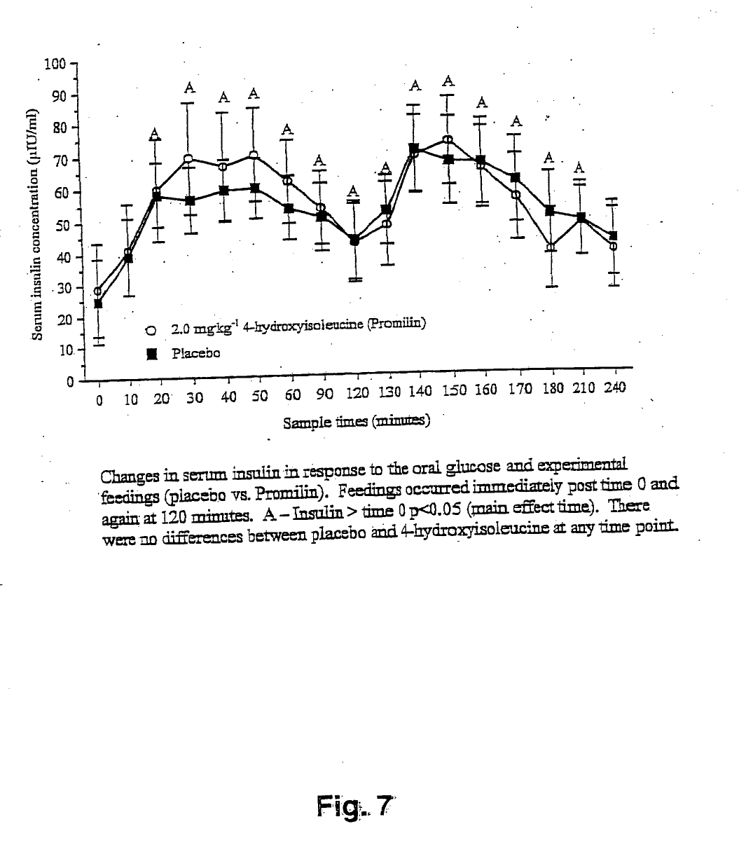

Referring now to FIG. 7, a graph is shown of the insulin concentration plotted over the time course of the post-exercise sample times. As illustrated, the vertical axis (i.e., ordinate) references serum insulin activity concentrations measured in micro International Unites per milliliter (μIU/mL) units. The horizontal axis (i.e., abscissa) references the sample times from time zero (0) (i.e., just after completing the glycogen depletion exercise and just before receiving glucose and the experimental feeding) to time 240 minutes. The confidence intervals for the plotted values are indicated by brackets. The data for the serum insulin response following placebo feedings are indicated by a solid box and the data for feedings with a novel composition of bio-active compounds derived, isolated, and/or extracted from fenugreek seeds are indicated by hollow circles. Those time points with an “A” indicate significant difference between the glucose level at the time point and the glucose level at time zero. The pattern of the curves are generally bi-modal and reflect the bolus administration of glucose and experimental feedings (i.e., a novel composition of bio-active compounds derived, isolated, and/or extracted from fenugreek seeds or placebo) at time zero and at time 120 minutes.

The results illustrated in FIG. 7 also indicate no significant differences in insulin concentration between the placebo and experimental trials. However, the main effect for time indicated a statistically significant increase in insulin from time zero (0) (i.e.,pre-feeding, immediately post exercise) at minutes twenty (20) and every time point thereafter until 240 minutes. By 240 minutes post initial feeding, serum insulin had returned to pre-feeding values.

Muscle Glycogen

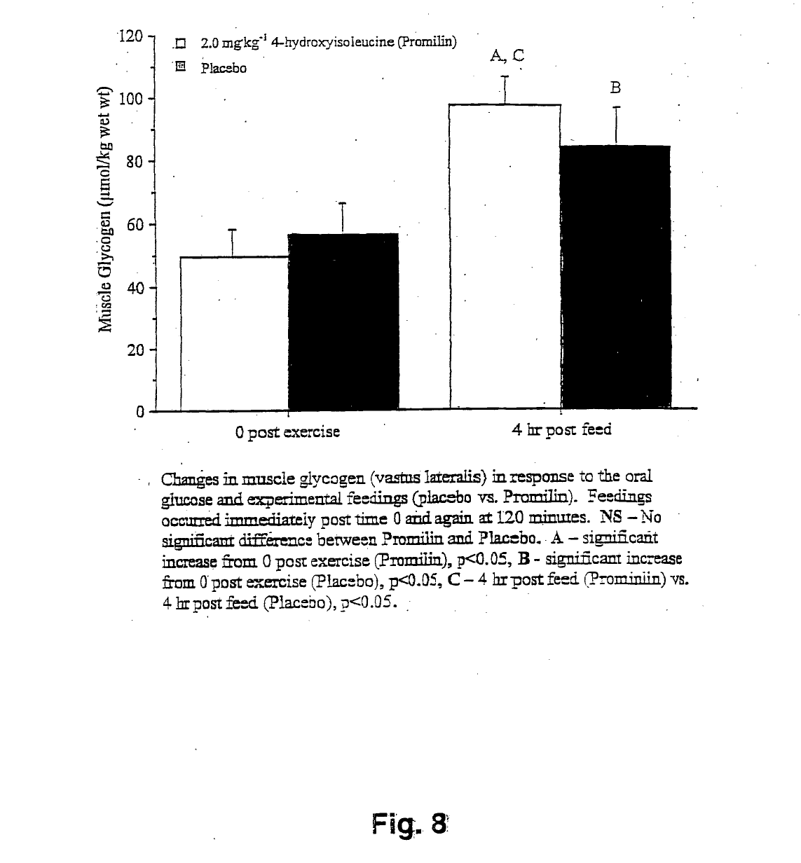

With reference now to FIG. 8, a chart is shown of the changes in muscle glycogen in the vastus lateralis muscle in response to the oral glucose and experimental feedings (i.e., a novel composition of bio-active compounds derived, isolated, and/or extracted from fenugreek seeds or placebo). As illustrated, the vertical axis (i.e., ordinate) references muscle glycogen in micro moles per kg of muscle weight (μmol/kg wet weight). The horizontal axis (i.e., abscissa) references time zero (i.e., immediately post-exercise and post-biopsy) and time 240 minutes (i.e., immediately after the second biopsy). Confidence intervals are indicated by the brackets extending above each bar. The solid bar represents the placebo data and the hollow bar represents the data for a novel composition of bio-active compounds derived, isolated, and/or extracted from fenugreek seeds.

As shown, the trial×time interaction for the measurement of muscle glycogen was significant indicating a difference in the rate of muscle glycogen resynthesis between the placebo and a composition of bio-active compounds derived, isolated, and/or extracted from fenugreek seeds. There was no significant difference immediately post exercise between the treatments showing equal glycogen depletion for both trials. Letters A and B indicate that both the placebo and of a presently preferred embodiment of a novel composition of bio-active compounds of the present invention demonstrated a significant increase in muscle glycogen following the four (4) hour recovery/feeding period (i.e., time zero to time 240 minutes). However, as indicated by letter C, at the end of the four (4) hour recovery/feeding period, muscle glycogen was significantly higher when the subjects received a composition of bio-active compounds of the present invention compared to when the subjects received the placebo.

Similarly calculated rates of muscle glycogen resynthesis were significantly higher during the experimental trial. Differences in the rates of muscle glycogen resynthesis (μmol/kg wet weight/hour) between the placebo and a novel composition of bio-active compounds derived, isolated, and/or extracted from fenugreek seeds are shown below in Table 1. Values were calculated from biopsy samples collected at zero (0) minutes post feeding and 240 minutes post feeding. Data are expressed as mean±one standard deviation (SD).

TABLE 1
Muscle glycogen resynthesis rate (μmol/kg wet weight/hr)

| | |
|---|---|
| Placebo | 6.7 ± 2.4 |
| Novel Composition of bio-active compounds | 11.1 ± 3.4* |
| derived, isolated, and/or extracted from | |
| fenugreek seeds | |

*p < 0.01 vs. placebo

Discussion

The foregoing Example demonstrates the effects of an oral preparation containing a composition which includes the active amino acid potentiator, 4-hydroxyisoleucine, in promoting the resynthesis of glycogen in muscle tissue. The main findings indicate that, in combination with a large oral carbohydrate bolus (i.e., 1.8 g/kg), a novel composition of bio-active compounds derived, isolated, and/or extracted from fenugreek seeds (based on 2.0 mg 4-hydroxyisoleucine/kg), promoted a forty percent (40%) higher rate of post-exercise muscle glycogen resynthesis compared to carbohydrate alone.

The tests conducted by the inventors demonstrate no significant differences in glucose or insulin concentrations between the carbohydrate+a novel composition of bio-active compounds derived, isolated, and/or extracted from fenugreek seeds and carbohydrate+placebo trials of the clinical example. Both trials demonstrate significant and sustained increases in glucose and insulin following both feedings (zero and 120 minutes) until returning to baseline values at 240 minutes.

One of the controversies regarding the acceleration of muscle glycogen resynthesis had included the potential to augment insulin release with supplemental protein sources. Although, the use of supplemental protein and/or amino acid in combination with oral glucose solutions in upwards of 1.8 g/kg may result in acute increase in insulin release, supplemental insulin does not typically accelerate exclusive glucose uptake by the depleted skeletal muscle. Moreover, the increase in insulin release may promote an increase in whole body glucose disposal and storage, stimulating glucose uptake by the liver, skeletal muscle, and adipose tissue. The lack of consistency regarding the effects of supplemental or amplified insulin release on accelerating muscle glycogen resynthesis suggests an upper physiological limit of glucose uptake by the skeletal muscle.

The rate of uptake and the concentration rate of glycogen resynthesis is likely a function of, but may not be limited to initial muscle glycogen concentration, total skeletal Glut-4 protein content, degree of Glut-4 translocation, and glycogen synthase activity. This foregoing Example suggests that insulin does not provide an additive effect to that promoted by skeletal muscle contraction. The present invention demonstrates a forty percent (40%) increase in the rate of muscle glycogen resynthesis with a novel composition of bio-active compounds derived, isolated, and/or extracted from fenugreek seeds without a significant difference in insulin or glucose across the treatments.

Other potential mechanisms may include enhanced insulin receptor activity or amplified Glut-4 translocation independent of muscle contraction and insulin. Regardless of the concept of multiple Glut-4 pools (insulin and contraction dependent), there is limited if any data to support the concept of Glut-4 translocation independent of the primary mechanisms of contraction and insulin.

Prior research has suggested that augmenting insulin release via the addition of specific amounts of protein and/or essential amino acids may further increase glucose uptake by the skeletal muscle and increase rates of glycogen resynthesis. However, the results gathered by the inventors demonstrate a forty percent (40%) increase in the rate of glycogen resynthesis with no significant increase in circulating insulin concentration and further suggests an additional mechanism to transport glucose into the muscle. As illustrated herein, this process is apparently enhanced by the combination of oral carbohydrate ingestion with a novel composition of bio-active compounds derived, isolated, and/or extracted from fenugreek seeds (based on 2.0 mg 4-hydroxyisoleucine/kg).

Additional embodiments and examples of the present invention may include quantification of the rates of muscle glycogen during the initial 120 minutes post feeding and the degree of muscle glycogen restoration following twenty-four (24) hours of controlled recovery. As Glut-4 activity has been shown to decrease in response to eccentric muscle damage, the addition of a novel composition of bio-active compounds derived, isolated, and/or extracted from fenugreek seeds with oral carbohydrate may enhance glycogen recovery in otherwise impaired skeletal muscle. The rate of glycogen recovery has also been linked with the maintenance of the functional synthetic rate and protein synthesis of skeletal muscle. Therefore the use of a novel composition of bio-active compounds derived, isolated, and/or extracted from fenugreek seeds may further enhance protein synthesis in response to vigorous muscle work and/or activity.

### EXAMPLE VIII

Metabolic Recovery, including muscle glycogen recovery, may be a critical component of exercise training, conditioning, and/or enhancement of athletic performance. Immediate post-exercise ingestion of a carbohydrate beverage may be required to enhance or maximize glycogen resynthesis in a mammal and such post-exercise ingestion may be integral to the recovery process. As appreciated by those skilled in the art, there is not a clear picture about ingestion of carbohydrates and protein in relation to metabolic recovery to date. Some studies suggest the importance of timing post-exercise and carbohydrate ingestion. In addition, some studies suggest protein ingestion combined with carbohydrates, or specific amino acids, to promote a more effective recovery time frame. Yet , there are other studies that contradicted these findings by demonstrating no improvement in recovery when proteins have been combined with carbohydrates in a post-exercise replenishment protocol.

One presently preferred embodiment of the novel compounds and methods of the present invention is contemplated to possess glucose-induced insulinotropic properties which may stimulate the pancreatic beta cells to produce more insulin in response to a glucose load. More importantly, novel compounds of the present invention may act independently of insulin by facilitating an enhanced signaling response via Glut-4. Both of these properties may potentially lead to faster metabolic recovery and recovery of muscle tissue from some level of endurance performance. In yet another presently preferred embodiment of the present invention, it is contemplated that a carbohydrate (CHO)/protein (PRO) beverage containing novel compounds of the present invention at each dose (i.e., high dose and low dose) improves recovery from intense interval running significantly more than a carbohydrate/protein beverage alone.

Methodology

Fifteen (15) well-conditioned athletes—eight (8) males and seven (7) females, ranging in age from 21-32 years, all of which are currently competitive at an elite level; nine (9) middle distance runners (800 m, 1500 m, triathletes), four (4) decathletes, and two (2) heptathletes—provided written consent prior to participation in a study directed to metabolic recovery. The group stratification was established by gender. Table 2 is a summary of the group characteristics.

TABLE 2
Subject Descriptive and Anthropometric Data (Mean ± SD)

| | |
|---|---|
| Number (N) | 15 |
| Age (year) | 27.0 ± 4.3 |
| Height (cm) | 180.0 ± 8.9 |
| Weight (Kg) | 81.1 ± 8.7 |
| Body Fat (%) | 14.0 ± 6.0 |
| Resting Heart Rate (BPM) | 46.0 ± 5.8 |
| Caloric Intake | |
| Range | 1500-4000 cal/day, not including |
| | compounds of the present invention |
| Mean | 2600 cal/day |
| Carbohydrate | 55%-70% |
| Protein | 15%-22% |
| Fats | 10%-18% |

The study participants were stratified and randomized into experimental and placebo groups. The experimental groups received either high dose (i.e., 0.3 g novel compounds of the present invention) or low dose (i.e., 0.15 g novel compounds of the present invention), whereas the placebo group received no compounds of the present invention. Participants in all three groups received carbohydrate and protein. All three treatments were in a powdered beverage matrix form which were subsequently mixed with thirty-two (32) ounces of tap water and shaken vigorously to create the post exercise beverage. The beverage was consumed in its entirety within thirty (30) minutes post exercise. The study was placebo-controlled and double-blinded. Tables 3 and 4 summarize the group stratification and dosages, respectively.

TABLE 3
Group stratification

| | | Number | Number of |
|---|---|---|---|
| N | Group | of Males | Females |
| 5 | High Dose | 3 | 2 |
| 5 | Low Dose | 2 | 3 |
| 5 | Placebo | 3 | 2 |

TABLE 4
Novel compounds of the invention,
Carbohydrate and Protein dosage

| | | Carbohydrate/Protein |
|---|---|---|
| Group (label) | Dose | Dose |
| High Dose (PT × 1) | 0.30 g novel | 60.0 g/24.7 g CHO/PRO |
| | compounds of | |
| | the invention | |
| Low Dose (PT × 2) | 0.15 g novel | 60.0 g/24.7 g CHO/PRO |
| | compounds of | |
| | the invention | |
| Placebo (PT × 3) | 0.00 g novel | 60.0 g/24.7 g CHO/PRO |
| | compounds of | |
| | the invention | |

All participants underwent testing with a Myotonometer® which was used to establish a baseline of muscle tone and stiffness at rest and at maximal contraction.

A baseline heart rate index was also determined; the formula heart rate index (HRI) is well established as [resting heart rate+exercise heart rate (1 min. step test)+30 sec. post+60 sec. post=heart rate index (HRI)]. A daily heart rate index number was taken with the criteria for recovery noted as follows: An increase of 0-10 beats per minute (BPM)=fully recovered, 11-20 BPM=not fully recovered, and 21+BPM=no recovery (should not train that day).

Statistical measurements were collected as standard error of measurement calculated for each force measurement to represent the standard deviation of measurement errors (ANOVA). Statistical significance was established using an alpha level of p<0.05 and p<0.01 for all measurements.

As illustrated in Table 5, the performance standards are summarized.

TABLE 5
Performance Standards.

| | | | Time | Time | Time |
|---|---|---|---|---|---|
| | Distance | | (seconds) | (seconds) | (minutes) |
| Trial | (meters) | | Male | Female | Rest |
| 1 | 1200 | 6 × 200 | 28-30 | 32-34 | 1.5 |
| 2 | 1400 | 7 × 200 | 28-30 | 32-34 | 1.5 |
| 3 | 1600 | 4 × 400 | 62-72 | 70-78 | 2.0 |
| 4 | 1800 | 6 × 300 | 39-42 | 42-48 | 2.0 |
| 5 | 2000 | 5 × 400 | 72-92 | 80-90 | 2.0 |
| 6 | 1200 | 4 × 300 | 39-42 | 42-48 | 2.0 |
| 7 | 1400 | 7 × 200 | 28-30 | 32-34 | 1.5 |
| 8 | 1600 | 4 × 400 | 60-70 | 70-78 | 2.0 |
| 9 | 1800 | 3 × 600 | 120-132 | 132-144 | 4.0 |
| | | 2 × 400 | 62-72 | 70-78 | 3.0 |
| 10 | 2000 | 2 × 200 | 35 | 40 | 2.0 |
| | | 4 × 100 | 14-16 | 16-18 | 1.5 |
| | | 1 × 400 | jog | jog | — |

The compositions administered during the study are summarized in Tables 6-8.

TABLE 6
Product Name: Promilin ™ Clinical A Study Code: (PT × 3)

| Ingredients | Amt/Svg (g) | % in Formula |
|---|---|---|
| Dextrose | 45 | 52.63 |
| Maltodextrin | 15 | 17.54 |
| Whey Protein Concentrate 80% | 19.72 | 23.07 |
| L-Leucine | 2.5 | 2.92 |
| L-Isoleucine | 1.25 | 1.46 |
| L-Valine | 1.25 | 1.46 |
| Promilin | 0 | 0.00 |
| Sodium Chloride | 0.635 | 0.74 |
| Magnesium Oxide | 0.14 | 0.16 |
| Flavor, Acid, Color, Sweetener | — | 0.00 |
| | 85.50 | 100 |

TABLE 7
Product Name: Promilin ™ Clinical B Study Code: (PT × 1)

| Ingredients | Amt/Svg (g) | % in Formula |
|---|---|---|
| Dextrose | 45 | 52.45 |
| Maltodextrin | 15 | 17.48 |
| Whey Protein Concentrate 80% | 19.72 | 22.99 |
| L-Leucine | 2.5 | 2.91 |
| L-Isoleucine | 1.25 | 1.46 |
| L-Valine | 1.25 | 1.46 |
| Promilin | 0.3 | 0.35 |
| Sodium Chloride | 0.635 | 0.74 |
| Magnesium Oxide | 0.14 | 0.16 |
| Flavor, Acid, Color, Sweetener | — | 0.00 |
| | 85.80 | 100 |

TABLE 8
Product Name: Promilin ™ Clinical C Study Code: (PT × 2)

| Ingredients | Amt/Svg (g) | % in Formula |
|---|---|---|
| Dextrose | 45 | 52.54 |
| Maltodextrin | 15 | 17.51 |
| Whey Protein Concentrate 80% | 19.72 | 23.03 |
| L-Leucine | 2.5 | 2.92 |
| L-Isoleucine | 1.25 | 1.46 |
| L-Valine | 1.25 | 1.46 |
| Promilin | 0.15 | 0.18 |
| Sodium Chloride | 0.635 | 0.74 |
| Magnesium Oxide | 0.14 | 0.16 |
| Flavor, Acid, Color, Sweetener | — | 0.00 |
| | 85.65 | 100 |

In each of the compositions in this present example, novel compounds of the present invention that were used were identified as Lot Number 2090898. The specification and certificate of analysis for Lot Number 2090898 is summarized in Table 9.

TABLE 9
Promilin ™-Certificate of Analysis. Lot No. 2090898^{†}

| Item | Specification | Result |
|---|---|---|
| Appearance | Brown fine powder | Complies |
| Odor | Characteristic | Complies |
| Assay (4-Hydroxyisoleucine) | Min. 20% | 26.2% |
| Residue on ignition | Max. 5.0% | 1.36% |
| Moisture (KF) | Max. 6.0% | 2.2% |
| Bulk density | Min. 0.4 g/mL | 0.61 g/mL |
| Heavy metals | Max. 10 ppm | Complies |
| Ash | Max. 2 ppm | Complies |
| Total anerobic microbial count | Max. 5,000 cfu/g | 50 cfu/g |
| Yeast & Mold | Max. 1,000 cfu/g | <10 cfu/g |
| Salmonella | Negative | Negative |
| E. Coli | Negative | Negative |
| Particle Size | NLT 90% through 40 | 94.55% |
| | mesh | |

^{†}Manufacture date: Sep. 6, 2002; Expiration date: Sep. 5, 2004; Analysis date: Sep. 27, 2002

In addition, dietary analysis software was employed in order to monitor each participant's daily dietary intake. This surveillance was undertaken to minimize or prevent outside influences, such as abnormally high caloric intakes of carbohydrate or protein, on the data being collected. Caloric intake and type of calories (i.e., carbohydrate, protein, etc.) were recorded on a daily basis for each participant. Additionally, subjects reported general health and well being comments such as energy levels, sleeping habits, perceived recovery, tiredness, etc. This information was logged daily along with the dietary intake.

Each study participant also was asked to complete a questionnaire to evaluate their subjective impression of the treatment. The questionnaire is outlined herein, as follows:

PROMILIN ™ QUESTIONNAIRE

```
  1. What is your knowledge or understanding level of the product
  Promilin ™?
```

| | | | | |
|---|---|---|---|---|
| 1. None | 2. Very | 3. Basic | 4. Very Good | 5. Great |
| | little | | | |

```
  2. What is your typical behavior post workout as it pertains to attempted
  recovery by ingestion of recovery drink?
```

| | | | | |
|---|---|---|---|---|
| 1. Never | 2. 30% | 3. 50% time | 4. 75% time | 5. 100% |
| | time | | | |

```
  3. What % recovery did you feel immediately post workout with
  ingestion of Promilin ™ (post < 30 min)?
```

| | | | | |
|---|---|---|---|---|
| 1. None | 2. 30% | 3. 50% | 4. 75% | 5. 100% |
| 0% | | | | |

4. What % recovery did you feel 2 hours post Promilin ™ ingestion?

| | | | | |
|---|---|---|---|---|
| 1. None | 2. 30% | 3. 50% | 4. 75% | 5. 100% |
| 0% | | | | |

5. What % recovery did you feel the following day?

| | | | | |
|---|---|---|---|---|
| 1. None | 2. 30% | 3. 50% | 4. 75% | 5. 100% |
| 0% | | | | |

6. Did you feel any side effects at any time after ingesting Promilin ™?

| | | | | |
|---|---|---|---|---|
| 1. None | 2. Slight | 3. Definite | 4. A lot | 5. Totally |
| | | | | un- |
| | | | | comfort- |
| | | | | able |
| Comment: | | | | |

```
  7. Did you or anyone living with you notice any offensive odor around
  you or the bathroom during the study?
```

| | | | | |
|---|---|---|---|---|
| 1. None | 2. Slight | 3. Definite | 4. A lot | 5. Totally |
| | | | | offens- |
| | | | | ive |

8. How would you rate your performance during training trials?

| | | | | |
|---|---|---|---|---|
| 1. Poor | 2. Less | 3. Average | 4. Above | 5. Great |
| | than | | average | |
| | average | | | |

9. Did your performance improve during the training trials?

| | | | | |
|---|---|---|---|---|
| 1. No | 2. Slightly | 3. Yes | 4. Greatly | 5. New PRs |

10. If Promilin ™ was offered commercially would you purchase it
regularly?

| | |
|---|---|
| 1. Yes- | 2. No- |
| Comment | Comment |

Experimental Protocol

Participants recorded heart rate index measurements each morning of the trial upon waking from rest. Next, they reported to the 400 meter track. After a series of warm-up exercises, stretching, and warm-up running, the performance trial began. The pre-trial measurements with the Myotonometer® were taken on the left leg of each subject. With the subject (i.e., participant) in the supine position, both relaxed and activated readings were taken of the vastus lateralis. The subjects were switched to a prone position to obtain relaxed and activated readings of lateral head of the left gastrocnemius.

To determine the placement of the probe, a dot corresponding to the center of the probe was drawn on the skin approximately in the mid-belly region of each muscle. For the vastus lateralis, a measurement midway between the greater trochanter of the femur and superior head of the fibula was established. For the lateral head of the gastrocnemius, the bony landmarks were the superior head of the fibula and inferior head of the same or the inferior aspect of the lateral maleolus. This distance was divided into thirds. One-third distance was then measured from the superior head of the fibula inferiorly, thus determining the point of measure.

Each muscle was tested in a relaxed state and during maximal voluntary contraction. The lateralis was tested at terminal extension both for the relaxed and the contracted measures. The relaxed state of the gastrocnemius was measured with the subject in the prone position; the contracted state of the gastrocnemius was also measured in the prone position while being contracted in total plantar flexion against another individual's leg while lying on the plinth, feet slightly over-hung.

Measurements were repeated every thirty (30) minutes post-exercise until pre-trial baseline was reached or two (2) hours time had elapsed, whichever came first. The measurements included five (5) probes of each muscle in both a relaxed and activated state. Pre- and post-protocol measurements were identical (5 probes). Following the performance trials and the thirty (30) minute post-trial Myotonometer® readings, the participants were allowed to walk around in an active recovery mode while awaiting subsequent measures. Once the time trials were completed for the day, the subject ingested the recovery beverage in its entirety within thirty (30) minutes post exercise.

The Myotonometer® is a proprietary electronic device that quantifies the amount of tissue displacement per unit force applied by a probe as it is pressed onto the skin overlying a muscle. The probe consists of an outer cylinder that remains stationary as an inner cylinder pushes onto and compresses underlying tissue. The distance between the outer and inner cylinders determines tissue displacement. The inner cylinder houses a force transducer that measures the amount of tissue resistance as the probe compresses the underlying tissue. Eight displacement measurements corresponding to 8 increments of force (0.25, 0.50, 0.75, 1.00, 1.25, 1.50, 1.75, 2.00 Kg) are obtained. Computational software generates force displacement curves based on these data. A more compliant (lower tone) muscle will have more displacement per unit force than a muscle with less compliance (higher tone). As appreciated, the Myotonometer® can assess any surface muscle in the body.

Discussion

The purpose of the present study was to evaluate dose levels and determine efficacy in recovery following intense performance trials with novel compounds of the present invention plus a CHO/PRO matrix vs. a CHO/PRO matrix alone or control. Three measurements were utilized to assess post-exercise muscle recovery: (1) Myotonometer® (muscle recovery); (2) heart rate index (cardiovascular recovery); and (3) running time trials (performance). Additionally, daily dietary intakes and general health comments were logged into dietary analysis software. Lastly, each subject filled out a questionnaire on the final testing day.

In the high dose group, the muscle recovery was statistically different than placebo. The Myotonometer® data demonstrated that eighty percent (80%) of high dose group recovered within a two-hour time frame versus only thirty-eight (38%) of the placebo group (5.4±2.1 vs. 10.8±3.1 mm/kg, respectively; p<0.05). Secondly, cardiovascular recovery, as evaluated by the heart rate index, showed over eighty percent (80%) of the subjects to be in the range of 0-10 BPM, meaning the fully recovered range, versus fifty-one percent (51%) of the subjects in the placebo group (260±6.3 vs. 290±11.8, respectively; p<0.01). Lastly, all participants (i.e., 100%) in the high dose group met or exceeded the performance times allowed for each running trial as compared to thirty percent (30%) in the placebo group (p<0.05).

The low dose group had similar, but somewhat less dramatic results than the high dose group. The Myotonometer® data demonstrated that sixty percent (60%) of the participants achieved baseline muscle recovery within two (2) hours versus thirty-eight percent (38%) of the placebo group (6.1±2.3 vs. 10.8±3.1 mm/kg, respectively; p<0.01). The heart rate index was slightly lower at seventy-three percent (73%) as compared to fifty-one percent (51%) for the placebo group (271±6.5 vs. 290±11.8, p<0.01). One female participant in the low dose group was not sleeping as well due to her child being ill. This may or may not have influenced lower heart rate index percentage. Performance measures were met sixty-six percent (66%) of the time in the low dose group versus thirty percent (30%) for the placebo group (p<0.05). Interestingly, members of the high dose group also reported better sleeping habits and a generally elevated energy level on a daily basis. Two people in the lower dose group reported the same in their daily logs, compared to the placebo group who reported a lack of recovery and/or chronic tiredness.

Based on the completed participant questionnaires, the majority of the individuals in the low and high dose groups felt the post recovery beverage containing novel compounds of the present invention was doing something for their performance and recovery, whereas the placebo group individuals remained less convinced. No side effects were reported during the trial or on the questionnaires. Two out of the fifteen individuals noticed a slight maple syrup odor.

Conclusion

Novel compounds of the present invention at the higher dose clearly demonstrated a significant result in all three areas evaluated, as did the lower dose with somewhat less magnitude when compared to the placebo group. In conclusion, the matrix of novel compounds of the present invention, together with CHO/PRO promotes an increased rate of recovery when ingested immediately after (i.e., within thirty (30) minutes following) a high intensity running interval workout. The elite athletes participating in the experimental trial became believers in the combination novel compounds of the present invention/CHO/PRO matrix as a recovery tool and are currently seeking the product. It is further contemplated that individuals from a more general athletic population, such as people who exercise less intensely but still need to recover fully to maximize their next effort, would also benefit from use of the novel compositions and methods of the present invention.

Other embodiments of the present invention may be directed to dose-dependent properties of a novel composition of bio-active compounds derived, isolated, and/or extracted from fenugreek seeds in glycogen recovery and/or synthesis, type and dose of carbohydrate used in combination with a novel composition of bio-active compounds derived, isolated, and/or extracted from fenugreek seeds, and the timing of administration relative to muscle work and/or glycogen depletion (i.e., prior, during, or following depletion).

Since the novel compositions of bio-active compounds derived, isolated, and/or extracted from fenugreek seeds of the present invention are configured to support metabolism and the transportation of glucose and other carbohydrates in animals and humans, it will be readily appreciated that a composition of bio-active compounds may contain 4-hydroxyisoleucine and one or more various bio-active compounds as described herein. Moreover, novel compositions of bio-active compounds of the present invention, may promote any number of physiological responses to administration of glucose or other carbohydrates, for example, but not by way of limitation, increased gut absorption of glucose, stimulation of pancreatic beta cells, enhanced glucose disposal from the blood.

It is also readily appreciated that the tests conducted on the human male subjects to evaluate the results of administration of a composition of bio-active compounds derived, isolated, and/or extracted from fenugreek seed extract (e.g., Promilin™ as used herein) to promote glycogen synthesis in muscle may be configured or modified to apply to any number of embodiments for practicing the present invention which are consistent with the spirit and scope of the present invention. It is intended, therefore, that the examples provided herein be viewed as exemplary of the principles of the present invention, and not as restrictive to a particular structure, method, process, or technique for implementing those principles.

In one presently preferred embodiment of the present invention, particular emphasis is placed on a composition of bio-active compounds including, for example, 4-hydroxyisoleucine, arginine, aspartate, threonine, serine, glutamate, glycine, alanine, cysteine, valine, methionine, isoleucine, leucine, phenylalanine, ornithine, lysine, histidine, and tyrosine. In one alternate embodiment of the present invention, particular emphasis may be placed on a composition of bio-active compounds including, for example, 4-hydroxyisoleucine, arginine, aspartate, threonine, serine, glutamate, proline, glycine, alanine, cysteine, valine, methionine, isoleucine, leucine, tryptophan, phenylalanine, ornithine, lysine, histidine and gamma-aminobutyrate.

In yet another alternative embodiment of the present invention, particular emphasis may be placed on a composition of bio-active compounds including, for example, 4-hydroxyisoleucine, arginine, aspartate, threonine, serine, glutamate, glycine, alanine, cysteine, valine, methionine, isoleucine, leucine, tryptophan, phenylalanine, ornithine, lysine, histidine and gamma-aminobutyrate. Further, in another presently preferred embodiment of the present invention, particular emphasis may be placed on a composition of bio-active compounds including, for example, 4-hydroxyisoleucine, arginine, aspartate, threonine, serine, glutamate, proline, glycine, alanine, cysteine, valine, methionine, isoleucine, leucine, tryptophan, phenylalanine, lysine, histidine and tyrosine.

Moreover, particular emphasis may be placed on a composition of bio-active compounds including, for example, 4-hydroxyisoleucine and one or more compounds selected from arginine, aspartate, threonine, serine, glutamate, proline, glycine, alanine, cysteine, valine, methionine, isoleucine, leucine, tryptophan, phenylalanine, proline, ornithine, lysine, histidine, gamma-aminobutyrate and tyrosine.

As further contemplated herein, novel compositions of bio-active compounds extracted from fenugreek seeds, which contain 4-hydroxyisoleucine and an array of one or more other amino acids may be combined with glucose or other carbohydrates, insulin, or insulin response modifiers to alter the physiological responses associated with administration of glucose or other carbohydrates, or to produce unique physiological responses. Physiological responses may include an increase in gut absorption of glucose, stimulation of pancreatic beta cells and enhanced disposal of glucose or other carbohydrates, or stimulation of glycogen synthesis (i.e., glycogenesis) in muscle, liver, and other areas in the bodies of mammals.

In addition, it is contemplated herein that, novel compositions of bio-active compounds extracted from fenugreek seeds of the present invention, which contain 4-hydroxyisoleucine and an array of one or more other amino acids may be combined with muscle growth supplements and administered to humans and other animals. Such combinations and methods may be used to promote muscle growth, promote a more lean body mass, or promote disposal of glucose and other carbohydrates from the blood.

Any number of muscle growth supplement compounds may be used to promote muscle growth and may include branched chain amino acids, growth hormone, whey protein, casein protein, creatine, glutamine, androstenedione, and dehydroepiandrosterone (DHEA). It may also be advantageous to combine one or more muscle growth supplements with novel compositions and methods of bio-active compounds from fenugreek seeds.

It will be further appreciated that the novel compositions of bio-active compounds derived, isolated, and/or extracted from fenugreek seeds of the present invention may be administered orally, parenterally, sublingual, topical, transdermal, intramuscular, or inhalation, and may also contain excipients chosen in accordance with the dosage form adopted. Moreover, the dosage of the extract compositions given to an individual may vary on the basis of several considerations without departing from the spirit and scope of the present invention and will, accordingly, depend on the targeted individual's particular case to be treated.

Also contemplated within the scope of the invention, novel compositions of bio-active compounds derived, isolated, and/or extracted from fenugreek seeds of the present invention may used to promote muscle function in a mammal, wherein muscle function may include metabolic recovery, muscle recovery, muscle growth, muscle contraction, muscle relaxation, enhancing glucose disposal, enhancing glucose absorption and enhancing glucose transport. Furthermore, methods for enhancing physiology of a mammal may include administering an effective amount of 4-hydroxyisoleucine for promoting muscle function in a mammal, wherein muscle function may include metabolic recovery, muscle recovery, muscle growth, muscle contraction, muscle relaxation, enhancing glucose disposal, enhancing glucose absorption and enhancing glucose transport.

From the above discussion, it will be appreciated that the present invention provides novel compositions and methods of bio-active compounds derived, isolated, and/or extracted from fenugreek seeds which are capable of facilitating an increase in the rate of glycogen synthesis in muscle, liver, and other area of mammals. Also, it will be appreciated that the present invention provides novel compositions and methods of bio-active compounds derived, isolated, and/or extracted from fenugreek seeds which are capable of stimulating the function of glucose transport factor 4 (Glut-4). In preferred design, the novel compositions and methods of bio-active compounds derived, isolated, and/or extracted from fenugreek seeds produce a high potency and quality extract yield that is economical and efficient to produce.

Unlike the prior art, the present invention provides novel compositions and methods of bio-active compounds derived, isolated, and/or extracted from fenugreek seeds including, without limitation, 4-hydroxyisoleucine, and one or more of the following: arginine, aspartate, threonine, serine, glutamate, proline, glycine, alanine, cysteine, valine, methionine, isoleucine, leucine, tryptophan, phenylalanine, ornithine, lysine, histidine, gamma-aminobutyrate, and tyrosine. As the studies and trials support, these novel extracted compositions from fenugreek seeds are capable of independently stimulating glucose transport proteins and facilitating the transport of glucose into muscles for the purpose of glycogen synthesis or, in the alternative, to work synergistically with insulin to stimulate glucose transport proteins and facilitate the transport of glucose into muscles or a direct effect on glycogen synthesis in muscle, liver, and other areas of the body.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

1. A process for promoting glycogen synthesis, comprising the step of administering an effective amount of 4-hydroxyisoleucine.

2. The process as defined in claim 1, further comprising the step of administering an effective amount of one or more amino acids selected from the group consisting of arginine, aspartate, threonine, serine, glutamate, proline, glycine, alanine, cysteine, valine, methionine, isoleucine, leucine, tryptophan, phenylalanine, ornithine, lysine, histidine, gamma-aminobutyrate, and tyrosine in combination with the 4-hydroxyisoleucine.

3. The process as defined in claim 2, wherein the combination is extracted from fenugreek seeds (Trigonella foenum graecum).

4. The process as defined in claim 2, wherein the combination comprises between about ten percent (10%) and about ninety percent (90%) amino acids and chemical salts, anhydrides, or isomers thereof.

5. The process as defined in claim 2, wherein the combination comprises between about ten percent (10%) and about seventy percent (70%) of the 4-hydroxyisoleucine and between about twenty percent (20%) and about forty percent (40%) of the amino acids and chemical salts, anhydrides, or isomers thereof.

6. The process as defined in claim 1, further comprising the step of administering an effective amount of glutamate in combination with the 4-hydroxyisoleucine.

7. The process as defined in claim 1, further comprising the step of administering an effective amount of glutamate and aspartate in combination with the 4-hydroxyisoleucine.

8. The process as defined in claim 1, further comprising the step of administering an effective amount of arginine, aspartate, threonine, serine, glutamate, glycine, alanine, cysteine, valine, methionine, isoleucine, leucine, phenylalanine, lysine, and histidine in combination with the 4-hydroxyisoleucine.

9. The process as defined in claim 1, further comprising the step of administering a carbohydrate in combination with the 4-hydroxyisoleucine.

10. The process as defined in claim 9, wherein the carbohydrate comprises at least one sugar selected from the group consisting of glucose, galactose, fructose, mannose, ribose, ribulose, arabinose, xylose, lyxose, and xylulose.

11. The process as defined in claim 1, further comprising the step of promoting insulin-independent glycogen synthesis.

12. The process as defined in claim 1, further comprising the step of promoting glycogen synthesis independent of an insulin-mediated pathway.

13. (canceled)

14. (canceled)

15. A process for promoting muscle function in mammals, comprising the step of administering a combination of 4-hydroxyisoleucine with an effective amount of one or more amino acids selected from the group consisting of arginine, aspartate, threonine, serine, glutamate, glycine, alanine, cysteine, valine, methionine, isoleucine, leucine, phenylalanine, lysine, and histidine.

16. The process as defined in claim 15, further comprising the step of administering an effective amount of one or more amino acids selected from the group consisting of proline, tryptophan, ornithine, gamma-aminobutyrate, and tyrosine.

17. The process as defined in claim 15, wherein the combination is extracted from fenugreek seeds (Trigonella foenum graecum).

18. The process as defined in claim 15, wherein the combination comprises between about ten percent (10%) and about ninety percent (90%) amino acids and chemical salts, anhydrides, or isomers thereof.

19. The process as defined in claim 15, wherein the combination comprises between about ten percent (10%) and about seventy percent (70%) of the 4-hydroxyisoleucine and between about twenty percent (20%) and about forty percent (40%) of the amino acids and chemical salts, anhydrides, or isomers thereof.

20. The process as defined in claim 15, further comprising the step of administering an effective amount of at least one muscle growth supplement selected from the group consisting of branched chain amino acids, arginine, lysine, methionine, histidine, growth hormone, whey protein, creatine, glutamine, androstenedione, and dehydroepiandrosterone.

21. The process as defined in claim 15, wherein the effective amount of one or more amino acids comprises glutamate.

22. The process as defined in claim 15, wherein the effective amount of one or more amino acids comprises glutamate and aspartate.

23. The process as defined in claim 15, further comprising the step of administering an effective amount of arginine, aspartate, threonine, serine, glutamate, glycine, alanine, cysteine, valine, methionine, isoleucine, leucine, phenylalanine, lysine, and histidine.

24. The process as defined in claim 15, further comprising the step of administering a carbohydrate.

25. The process as defined in claim 24, wherein the carbohydrate comprises at least one sugar selected from the group consisting of glucose, galactose, fructose, mannose, ribose, ribulose, arabinose, xylose, lyxose, and xylulose.

26. The process as defined in claim 15, wherein said muscle function is selected from the group consisting of metabolic recovery, muscle recovery, muscle growth, muscle contraction, muscle relaxation, enhancing glucose disposal, enhancing glucose absorption, and enhancing glucose transport.

27. (canceled)

28. The process as defined in claim 15, wherein the combination further comprises at least one insulin response modifier selected from the group consisting of insulin, insulin secretagogue, biguanide, alpha-glucosidase inhibitor, and thiazolidinediones.

29. The process as defined in claim 28, wherein the insulin secretagogue comprises a sulfonylurea compound.

30. The process as defined in claim 28, wherein the sulfonylurea compound is selected from the group consisting of tolbutamide, acetohexamide, tolazamide, chlorpropamide, glyburide, glipizide, glimepiride and analogues, isomers, or pharmacological salts thereof.

31. The process as defined in claim 28, wherein the biguanide further comprises metformin and analogues, isomers, or pharmacological salts thereof.

32. The process as defined in claim 29, wherein the alpha-glucosidase inhibitor further comprises acarbose and miglitol and analogues, isomers, or pharmacological salts thereof.

33. The process as defined in claim 15, further comprising the step of promoting insulin-independent muscle function.

34. The process as defined in claim 15, further comprising the step of promoting muscle function independent of an insulin-mediated pathway.

35-40. (canceled)

41. The process as defined in claim 1, further comprising the step of stimulating glucose transport proteins to translocate glucose into liver, muscle, kidney, or intestine.

42. The process as defined in claim 41, wherein the glucose transport proteins comprise glucose transport proteins types 1-7 (GT 1-7).

43. The process as defined in claim 41, wherein the glucose transport proteins comprise glucose transport protein type 4 (GT 4).

44. The process as defined in claim 1, further comprising the step of increasing serum insulin concentration within from about one (1) minute to about seventy-five (75) minutes following administration of 4-hydroxyisoleucine.

45. The process as defined in claim 1, further comprising the step of increasing serum insulin concentration within from about ten (10) minutes to about thirty (30) minutes following administration of 4-hydroxyisoleucine.

46. The process as defined in claim 1, further comprising the step of increasing serum insulin concentration within about fifteen (15) minutes following administration of 4-hydroxyisoleucine.

47. The process as defined in claim 1, wherein said step of administering is selected from the group consisting of oral, parenteral, sublingual, topical, transdermal, intramuscular, intranasal, and inhalation.

48. The process as defined in claim 1, wherein the effective amount of 4-hydroxyisoleucine is between about one (1) to about nine (9) mg 4-hydroxyisoleucine per kg of body weight.

49. The process as defined in claim 1, wherein the effective amount of 4-hydroxyisoleucine is about two (2) mg 4-hydroxyisoleucine per kg of body weight.

50-60. (canceled)

61. The process as defined in claim 15, further comprising the step of storing energy.

62. The process as defined in claim 61, wherein the step of energy storage comprises the step of stimulating glucose transport proteins to translocate glucose into muscle.

63. The process as defined in claim 62, wherein the glucose transport proteins comprise glucose transport proteins types 1-7 (GT 1-7).

64. The process as defined in claim 62, wherein the glucose transport proteins comprise glucose transport protein type 4 (GT 4).

65. The process as defined in claim 15, wherein the step of administering is selected from the group consisting of oral, parenteral, sublingual, topical, transdermal, intramuscular, intranasal, and inhalation.

66. The process as defined in claim 15, wherein the effective amount of 4-hydroxyisoleucine is between about one (1) to about nine (9) mg 4-hydroxyisoleucine per kg of body weight.

67. The process as defined in claim 15, wherein the effective amount of 4-hydroxyisoleucine is about two (2) mg 4-hydroxyisoleucine per kg of body weight.

68. (canceled)

69. (canceled)

70. The process as defined in claim 15, further comprising the step of administering an effective amount of at least one muscle growth supplements selected from the group consisting of branched chain amino acids, arginine, lysine, methionine, histidine, growth hormone, whey protein, casein protein, creatine, glutamine, androstenedione, and dehydroepiandrosterone.

71. (canceled)

72. (canceled)

73. The process as defined in claim 15, further comprises the step of administering an effective amount of at least one insulin response modifier selected from the group consisting of insulin, insulin secretagogue, biguanide, alpha-glucosidase inhibitor, and thiazolidinediones.

74. The process as defined in claim 73, wherein the insulin secretagogue comprises a sulfonylurea compound.

75. The process as defined in claim 74, wherein the sulfonylurea compound is selected from the group consisting of tolbutamide, acetohexamide, tolazamide, chlorpropamide, glyburide, glipizide, glimepiride and analogues, isomers, or pharmacological salts thereof.

76. The process as defined in claim 73, wherein the biguanide further comprises metformin and analogues, isomers, or pharmacological salts thereof.

77. The process as defined in claim 73, wherein the alpha-glucosidase inhibitor further comprises acarbose and miglitol and analogues, isomers, or pharmacological salts thereof.

78. (canceled)

79. (canceled)
